(19) United States
(12) Patent Application Publication
(10) Pub. No.: US 2025/0221981 A1
(43) Pub. Date: Jul. 10, 2025

(54) MAT2A INHIBITOR AND/OR TSG101 INHIBITOR FOR USE IN ANTIVIRAL THERAPY

(71) Applicant: CAMBRIDGE ENTERPRISE LIMITED, Cambridge, Cambridgeshire (GB)

(72) Inventors: Colin Michael CRUMP, Cambridge (GB); Laura Grace CALLER, Cambridge (GB)

(21) Appl. No.: 18/853,949

(22) PCT Filed: Mar. 31, 2023
(86) PCT No.: PCT/GB2023/050875

§ 371 Date: Oct. 3, 2024

(30) Foreign Application Priority Data
Apr. 4, 2022 (GB) 2204913.4

(51) Int. Cl. A61K 31/4745; A61K 31/4439; A61K 31/517; A61K 31/519; A61P 31/14; A61P 31/20; A61P 31/22

(52) U.S. Cl. CPC A61K 31/4745; A61K 31/4439; A61K 31/517; A61K 31/519; A61P 31/14; A61P 31/20; A61P 31/22

(57) ABSTRACT

The invention relates to MAT2A inhibitors and/or TSG101 inhibitors for treating or preventing viral infection in a subject. More specifically, the invention relates to MAT2A inhibitors and/or TSG101 inhibitors for treating or preventing viral infections caused by DNA viruses or nuclear replicating viruses. The invention also relates to MAT2A inhibitors and/or TSG101 inhibitors for treating or preventing viral infections caused by RNA viruses.

### FIELD OF THE INVENTION

The invention relates to MAT2A inhibitors and/or TSG101 inhibitors for treating or preventing viral infection in a subject. More specifically, the invention relates to MAT2A inhibitors and/or TSG101 inhibitors for treating or preventing viral infections caused by DNA viruses or by nuclear replicating viruses. The invention also relates to MAT2A inhibitors and/or TSG101 inhibitors for treating or preventing viral infections caused by RNA viruses.

### BACKGROUND OF THE INVENTION

Various types of viruses, such as nuclear replicating viruses and DNA viruses, rely on host cell machinery to achieve efficient viral gene expression and viral replication. DNA viruses include a range of important pathogens such as polyomaviruses, papillomaviruses, herpesviruses, poxviruses, and adenoviruses, and infections caused by these viruses present a major health concern worldwide. Many of these viruses are difficult to target with antivirals because they do not produce enzymes such as polymerases. Nuclear replicating viruses also include a range of important pathogens, including HIV and influenza.

Polyomaviruses are commonly found in the reno-urinary tract of humans, where they normally cause an asymptomatic infection. However, these viruses have the potential to cause serious disease in certain patient groups, e.g. in immunosuppressed patients. BK polyomavirus (BKPyV) is a major cause of polyomavirus associated nephropathy (PVAN) which can occur in up to 15% of kidney transplant patients and can result in graft failure. BKPyV is thought to contribute to premature graft failure in 1-15% of kidney transplant recipients. Despite the significant impact that BKPyV infections have on the success rate of kidney transplants, treatment options for PVAN are effectively limited to reduction of immune suppression which also increases the risks of transplant rejection. Approximately 100,000 kidney transplants occurred worldwide in 2019, and so prevention and/or treatment of PVAN and associated graft failure represents a significant global unmet clinical need.

BKPyV is also a common cause of late-onset haemorrhagic cystitis which is a well-recognised and significant complication that affects transplant patients, such as haematopoietic stem cell (HSC) transplant patients. Late-onset haemorrhagic cystitis increases the morbidity of these patients and also increases the mortality rate. Haemorrhagic cystitis can be treated with low dose cidofovir, but nephrotoxicity associated with this drug prevents widespread use. Approximately 20,000 HSC transplants occur in the US each year, and so effective prevention and/or management of late-onset haemorrhagic cystitis also represents a major unmet clinical need.

JC polyomavirus (JCPyV), which is closely related to BKPyV, can cause progressive multifocal leukoencephalopathy (PML) which is a rare but invariably fatal disease of the central nervous system that typically affects immunosuppressed patients, e.g. those suffering from HIV and/or those undergoing antibody (e.g. monoclonal antibody) therapy for a lymphoproliferative disease.

Polyomaviruses have also been implicated in cancer. For example, Merkel cell polyomavirus (MCPyV) is thought to be a causative factor in the majority of cases of the skin cancer Merkel cell carcinoma.

Polyomaviruses and papillomaviruses were previously categorised together in the same taxonomic family (Papovaviridae) until 1999 due in part to their similar morphology and genome organisation. Similar to polyomaviruses, papillomaviruses rely on host cell machinery for viral genome replication and essential splicing of viral mRNA. Human papillomavirus (HPV) infections are often asymptomatic, but can cause precancerous lesions and increase the risk of certain cancers. There are no treatments available for HPV, and current therapeutic interventions are focussed on vaccination.

Viruses, such as DNA viruses or nuclear replicating viruses, represent a major clinical concern, particularly in vulnerable patient groups, and there are limited treatment options available. There is therefore an urgent and significant unmet need for effective therapies to treat and/or prevent viral infections caused by such viruses.

### SUMMARY OF THE INVENTION

The Inventors have overcome the above problems by identifying key proteins that are produced by host cells and required by viruses, such as DNA viruses or nuclear replicating viruses, to establish early and late gene expression, and to replicate efficiently. Advantageously, inhibition of MAT2A and/or TSG101 was found to significantly impede viral replication within host cells, thereby providing a new and unexpected therapeutic strategy for treating and/or preventing viral infections caused by viruses, such as DNA viruses or nuclear replicating viruses.

The invention provides a MAT2A inhibitor and/or a TSG101 inhibitor for use in a method of treating or preventing a viral infection in a subject.

The invention also provides a method for treating or preventing a viral infection in a subject, the method comprising administering to the subject a MAT2A inhibitor and/or a TSG101 inhibitor.

The invention also provides use of a MAT2A inhibitor and/or a TSG101 inhibitor in the treatment or prevention of a viral infection in a subject.

In some embodiments, the viral infection is caused by a DNA virus. In some embodiments, the DNA virus is a double stranded DNA virus. In some embodiments, the DNA virus is selected from a polyomavirus, a papillomavirus, an adenovirus, a poxvirus, or a herpesvirus. In some embodiments, the DNA virus is selected from a polyomavirus, a papillomavirus, an adenovirus, or a herpesvirus. In some embodiments, the DNA virus is a polyomavirus. In some embodiments, the DNA virus is BK polyomavirus or JC polyomavirus.

In some embodiments, the method comprises treating or preventing polyomavirus associated nephropathy.

In some embodiments, the method comprises treating or preventing progressive multifocal leukoencephalopathy.

In some embodiments, the method comprises treating or preventing late-onset haemorrhagic cystitis.

In some embodiments, the DNA virus is a papillomavirus.

In some embodiments, the viral infection is caused by a nuclear replicating virus. In some embodiments, the nuclear replicating virus is a retrovirus, such as HIV. In some embodiments, the nuclear replicating virus is an influenza virus.

In some embodiments, the viral infection is caused by an RNA virus, optionally wherein the RNA virus is selected from an alphavirus, an astrovirus, a calicivirus, a flavivirus, and a picornavirus. In some embodiments, the RNA virus is selected from a Sindbis virus, a Semliki Forest virus and Zika virus.

In some embodiments, the viral infection is causes by a non-enveloped virus. In some embodiments, the non-enveloped virus is selected from a polyomavirus, a papillomavirus, an adenovirus, a parvovirus, an astrovirus, a calicivirus, a picornavirus, or a reovirus.

In some embodiments, the viral infection is causes by an enveloped virus.

In some embodiments, the invention provides a TSG101 inhibitor for use in a method of treating or preventing a viral infection in a subject, wherein the viral infection is caused by a non-enveloped virus. In some embodiments, the invention provides a TSG101 inhibitor for use in a method of treating or preventing a viral infection in a subject, wherein the viral infection is caused by an enveloped virus.

In some embodiments, the invention provides a MAT2A inhibitor for use in a method of treating or preventing a viral infection in a subject, wherein the viral infection is caused by a non-enveloped virus. In some embodiments, the invention provides a MAT2A inhibitor for use in a method of treating or preventing a viral infection in a subject, wherein the viral infection is caused by an enveloped virus.

In some embodiments, the subject is immunosuppressed. In some embodiments, the subject is a transplant patient. In some embodiments, the subject is a kidney transplant patient. In some embodiments, the subject is a haematopoietic stem cell transplant patient.

In some embodiments, the MAT2A inhibitor is selected from a small molecule, a peptide, a cyclic peptide, and a peptidomimetic.

In some embodiments, the MAT2A inhibitor is:

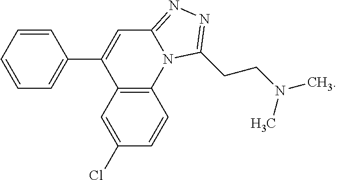 [SMILES: CN(C)CCc1nnc2cc(-c3ccccc3)c3cc(Cl)ccc3n12]

In some embodiments, the MAT2A inhibitor is:

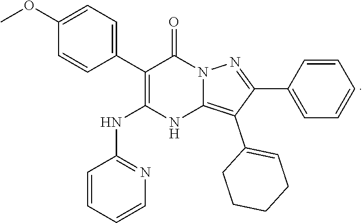 [SMILES: COc1ccc(-c2c(Nc3ccccn3)[nH]c3c(C4=CCCCC4)c(-c4ccccc4)nn3c2=O)cc1]

In some embodiments, the MAT2A inhibitor is:

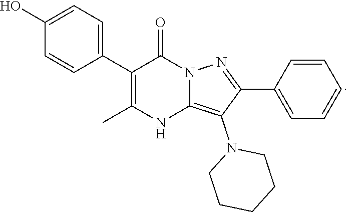 [SMILES: Cc1[nH]c2c(N3CCCCC3)c(-c3ccccc3)nn2c(=O)c1-c1ccc(O)cc1]

In some embodiments, the MAT2A inhibitor is:

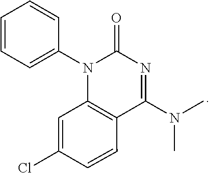 [SMILES: CN(C)c1nc(=O)n(-c2ccccc2)c2cc(Cl)ccc12]

In some embodiments, the MAT2A inhibitor is:

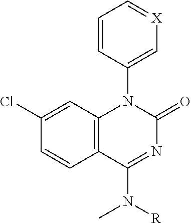

wherein R is Me or Et and X is C or N.

In some embodiments, the MAT2A inhibitor is:

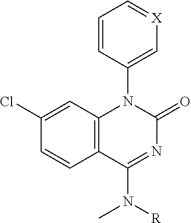

wherein:
(a) R is Me and X is C (referred to herein as AZ28);
(b) R is Me and X is N (referred to herein as AZ31);
(c) R is Et and X is C (referred to herein as AZ32); or
(d) R is Et and X is N (referred to herein as AZ33).

In some embodiments, the MAT2A inhibitor is IDE397.

In some embodiments, the MAT2A inhibitor is an oligonucleotide, optionally wherein the MAT2A inhibitor is an antisense oligonucleotide, a shRNA, a siRNA, a microRNA, or an aptamer.

In some embodiments, the MAT2A inhibitor is an antibody.

In some embodiments, the TSG101 inhibitor is selected from a small molecule, a peptide, a cyclic peptide, and a peptidomimetic.

In some embodiments, the TSG101 inhibitor is a prazole. In some embodiments, the TSG101 inhibitor is Ilaprazole. In some embodiments, the TSG101 inhibitor is selected from Ilaprazole, omeprazole, esomeprazole, dexlansoprazole, lansoprazole, pantoprazole, rabeprazole, and tenatoprazole. In some embodiments, the TSG101 inhibitor is Ilaprazole.

In some embodiments, the TSG101 inhibitor is selected from an oligonucleotide, optionally wherein the TSG101 inhibitor is an antisense oligonucleotide, a shRNA, a siRNA, a microRNA, or an aptamer.

In some embodiments, the TSG101 inhibitor is an antibody.

### BRIEF DESCRIPTION OF THE DRAWINGS

FIG. 1: Experimental design of a CRISPR/Cas9 genome-wide screen, identifying MAT2A as a gene important for BKPyV early viral gene expression.

(A) Experimental design of screen: Cas9-RPTE/TERT1 cells, a human renal cell line stably expressing Cas9, were used to conduct a genome-wide screen for factors important in BKPyV entry and early viral gene expression.

Cells were transduced with lentivirus comprising sgRNAs of the Bassik CRISPR library, in which there are 10 sgRNAs per gene for all ˜20,500 protein coding genes of the human genome, a puromycin resistance gene and an mCherry gene. Transduced cells were selected in puromycin for 8 days to select for cells which were successfully transduced. Puromycin selected cells were then BKPyV infected (MOI 5) or left uninfected to serve as the reference library. After 3 days infected cells were fixed, permeabilised and stained for viral early gene Large T Antigen (LTAg), followed by FACS sorting for mCherry positivity (gene transduced), and lowest 10% LTAg expression (reduced early viral gene expression).

Next generation sequencing was used to determine the enrichment of specific sgRNA sequences in DNA extracted from the sorted population compared to the unsorted cell population library.

(B) Plot illustrating the distribution of genes identified from the CRISPR/Cas9 screen: All genes are plotted alphabetically (x-axis) against enrichment significance in the lowest 10% of LTAg expressing cells (y-axis). Those genes over −Log (p-value) of 3 are considered significant. Genes ranked alphabetically; significance displayed as −Log (p-value). One of the most significantly enriched genes was MAT2A, highlighted in this plot. Significance p<0.00005, 9 of 10 MAT2A CRISPR sgRNAs identified.

FIG. 2: MAT2A knockout RPTE/TERT1 cells validate importance of MAT2A for viral gene expression, viral genome replication, and infectious virus production.

(A) Effect of MAT2A knockout on viral early gene expression: CRISPR knockout MAT2A single cell clones (RPTE/TERT1 MAT2A K/O) or parent RPTE/TERT1 cells were infected with BKPyV (MOI 3) and formaldehyde fixed at 48 hours post infection (hpi). Cells were stained for LTAg expression and detected by fluorescence microscopy. The number of infected LTAg positive cells were recorded (i), the mean nucleus size of those infected cells was measured (ii) and mean total LTAg fluorescence of infected cells was recorded (iii). All results normalized to parental control cells. n=3.

(B) Effect of MAT2A knockout on viral early and late gene expression: Parent and MAT2A K/O RPTE/TERT1 cells were infected with BKPyV (MOI 3) or mock infected. At 48 hpi cells were harvested and a western blot conducted and stained for LTAg, viral late gene VP1, cellular MAT2A/MAT1A and tubulin as a loading control. The MAT2A homolog MAT1A shows here as a faint band the same size as MAT2A.

(C) Effect of MAT2A knockout on viral genome replication: Parent and MAT2A K/O RPTE/TERT1 cells were infected with BKPyV (MOI 3). At 48 hpi cells were pelleted and qPCR conducted. BKPyV genomes per cell were quantified, using GAPDH as a control. Results normalised to parental control cells. n=3.

(D) Effect of MAT2A knockout on viral replication: Parent and MAT2A K/O RPTE/TERT1 cells were infected with BKPyV (MOI 5). At 24, 48, 72 and 96 hpi cells were harvested and infectious BKPyV was measured by fluorescence focus unit (FFU) assay. n=3 (*p<0.05, **p<0.01).

FIG. 3: Inhibition of MAT2A with PF-9366 confirms importance of MAT2A activity for BKPyV gene expression and replication.

RPTE/TERT1 cells were treated with PF-9366 at a range of concentrations, or DMSO treated as a control, for 3 hrs prior to infection.

(A) Effect of PF-9366 on viral early gene expression: Cells were infected with BKPyV (MOI 1) in media containing appropriate drug concentrations. At 1 hpi infectious media was removed, cells were washed and fresh media with appropriate drug concentrations was added. Cells were formaldehyde fixed at 48 hpi and stained for DAPI and LTAg expression and detected by fluorescence microscopy. The number of infected LTAg positive cells were recorded, normalized to cell number. n=3.

(B) Effect of PF-9366 on viral replication: Cells were infected with BKPyV (MOI 1) in media containing appropriate drug concentrations. At 1 hpi infectious media was removed, cells were washed and fresh media with appropriate drug concentrations was added. At 48 hpi cells were harvested and infectious BKPyV was measured by FFU assay. n=3.

(C)(i) Effect of PF-9366 on viral early and late gene and cellular MAT2A expression: Cells were infected with BKPyV (MOI 3) in media containing appropriate drug concentrations. At 1 hpi infectious media was removed, cells were washed and fresh media with appropriate drug concentrations was added. Cells were harvested at 48 hpi and a western blot performed. Membranes were blotted for viral late gene VP1, LTAg, cellular MAT2A/MAT1A, and tubulin as a loading control.

(ii) Cellular MAT2A expression increases at lower PF-9366 concentrations: The density of MAT2A expression (lower western blot band) was measured, normalized to tubulin loading control (LiCor Image Studio Software), n=3.

(iii) Viral gene expression is modulated in response to PF-9366: The density of BKPyV VP1 and LTAg expression were measured, normalized to tubulin loading control (LiCor Image Studio Software), n=3.

(*p≤0.05, **p≤0.01, ***p≤0.001, ***p≤0.0001).

FIG. 4: Inhibition of MAT2A with AG-270 reduces viral early and late gene expression.

RPTE/TERT1 cells were treated at proliferative IC50 PF-9366 (10 μM) or AG-270 (257 nM), double proliferative IC50 (PF-9366 20 μM, AG-270 514 nM) or DMSO treated as a control, for 3 hrs prior to infection.

Effect of drug treatment on cellular replication and survival and viral early gene expression: Cells were infected with BKPyV (MOI 1) in media containing appropriate drug concentrations. At 1 hpi infectious media was removed, cells were washed and fresh media with appropriate drug concentrations was added. Cells were formaldehyde fixed at 48 hpi and stained for nuclear DAPI and LTAg expression, then detected by fluorescence microscopy. The number of cells per well were determined through DAPI analysis (i) Effect of drug treatment on viral early and late gene expression: The number of infected cells were determined by LTAg (early gene) and VP1 (late gene) expression (ii).

FIG. 5: Cell viability assay of MAT2A inhibitors.

RPTE/hTERT cells were treated with MAT2A inhibitors at increasing concentrations for 48 hrs and assayed for cell viability using CellTiter-Blue fluorescence. Experiments were conducted using biological triplicates.

FIG. 6: Dose dependent effects of MAT2A inhibitors on total BKPyV virion synthesis and viral protein expression.

RPTE/hTERT cells were treated with different MAT2A inhibitors (AZ28, AZ31, AZ32, AZ33 and AG270) at an increasing concentration of 125 nM, 250 nM, 500 nM and 1 μM. Cells were treated with MAT2A inhibitors at 1 hr post infection and were harvested at 48 hr post infection at MOI 1. (A) Total infectious BKPyV synthesis expressed as fold change to DMSO control (FFU titre assay). (B) Total viral protein expression (LTAg and VP1) was analysed by western blot. Tubulin was used as loading control. All error bars represent SEM. Two sample paired T-test was used to determine significance (*p<0.05; **p<0.01; ***p<0.001, ****p<0.0001), all numerical data shown were obtained over 2 independent experiments.

FIG. 7: Cell viability assay of drug treated RPTE/hTERT cells.

Cells were treated with MAT2A inhibitors AZ33 or AG270, TSG101 inhibitor Ilaprazole, and combination of both Ilaprazole and MAT2A inhibitors and assayed for cell viability using CellTiter-Blue fluorescence every 48 hr until 144 hr. Experiment was conducted using biological triplicates.

FIG. 8: Effects of Ilaprazole and MAT2A inhibitors in combination on BKPyV replication.

RPTE/hTERT cells were treated with inhibitors at 1 hr post infection (PI), infected at MOI 1. (A) Viral growth curves using FFU titre assay in drug treated and untreated cells infected with BKPyV at MOI 1 for 48 hr PI, 72 hr PI and 96 hr PI. (B) Total viral protein expression (LTAg and VP1) from drug treatments and untreated control was analysed by western blot with samples harvested at 48 hr PI and 96 hr PI. Tubulin was used as loading control. All error bars represent SEM. All numerical data shown were obtained over 2 independent experiments.

FIG. 9: Inhibition of MAT2A with PF-9366 reduces Herpes Simplex Virus-1 (HSV-1) virion assembly Immortalised keratinocyte (HaCaT) cells were plated and treated with 20 μM PF-9366 or DMSO control. After 3 hrs cells were infected with HSV-1 (MOI 10). After 16 hrs cells were harvested by scraping and either pelleted for western blot or freeze/thawed for titration. To titre: VERO cells were plated and the following day infected with 10-fold dilutions, at 1 hpi cells were overlayed with CMC/media. At 48 hpi cells were fixed and stained with toluidine blue. To western blot: Pellets were lysed and run on SDS-PAGE gel. Proteins were transferred overnight to nitrocellulose membranes. Membranes were probed for HSV-1 ICP0 and VP16 proteins and Tubulin as a control.

FIG. 10: Sindbis virus (SINV), Semliki Forest Virus 4 (SFV4) and Zika virus show a reduction in virus replication and/or viral protein expression when treated with MAT2A inhibitor PF-9366.

(A) SINV and SFV4: BHK-21 cells were plated. The following day 2 μM PF-9366 or DMSO control was added to media. After 3 hrs cells were infected with SINV or SFV4 (MOI >10). After 24 hrs cells were harvested by scraping. Cells were either pelleted for western blot or freeze/thawed for titration. To titre: BSR cells were plated and the following day infected with 10-fold dilutions, at 1 hpi cells were overlayed with LMA in 2×MEM (+5% FCS). At 72 hpi cells were fixed and stained with toluidine blue.

(B) Zika: VERO E6 cells were plated. The following day 20 μM PF-9366 or DMSO control was added to media. After 3 hrs cells were infected with Zika African strain, American Strain or Mock infected (MOI >10). After 48 hrs cells were harvested by scraping, pelleted and lysed into lysis buffer. Western blot was then conducted on the lysed cells. Protein embedded SDS-PAGE gels were transferred to membranes overnight and then probed for Zika E protein and GAPDH as a control.

### DETAILED DESCRIPTION OF THE INVENTION

To identify genes that are important for virus early gene expression in human cells, the Inventors performed a CRISPR-Cas9 whole genome screen. Using the DNA virus BKPyV as a model system, the Inventors identified that the expression of the early viral gene large tumour antigen (LTAg) was significantly reduced when expression of MAT2A was disrupted. To validate the importance of MAT2A for BKPyV infection, the Inventors generated a MAT2A gene knockout cell line. Surprisingly, the Inventors discovered that expression of both early (LTAg) and late (major capsid protein VP1) viral genes were significantly reduced in the MAT2A knockout cell line. The Inventors also found that BKPyV replication was significantly reduced in the absence of MAT2A.

To confirm that MAT2A inhibition was responsible for reducing viral replication, the Inventors tested whether structurally distinct MAT2A inhibitors (PF-9366, AG-270, AZ28, AZ31, AZ32 and AZ33) could inhibit viral gene expression and replication. The expression of both early and late viral genes was significantly reduced in the presence of all of the MAT2A inhibitors tested. In addition, the Inventors demonstrated that viral replication was significantly reduced in the presence of MAT2A inhibitors. These results demonstrate the importance of MAT2A activity in virus gene expression and replication.

MAT2A (S-adenosylmethionine synthase isoform type-2, also known as Methionine Adenosyltransferase 2A) is an enzyme which catalyses S-adenosylmethionine (SAM) formation from methionine and ATP. SAM is an essential factor for methylation reactions which are involved in various cellular functions including RNA metabolism, gene expression (transcription and translation), and genome replication. DNA viruses and nuclear replicating viruses often have a strict dependence on the host cell DNA replication machinery and associated cell cycle regulation, as well as RNA splicing, to achieve productive infection. MAT2A plays an important role in the host cell processes on which DNA viruses and nuclear replicating viruses rely and, based on the results described herein, these types of viruses are expected to be susceptible to MAT2A inhibition.

Surprisingly, the Inventors also discovered that MAT2A inhibitors can reduce virus titre and viral protein expression of RNA viruses, such as alphaviruses, astroviruses, caliciviruses, flaviviruses and picornaviruses. Methylation of viral genomic RNA and mRNA, such as methylation at the N6 position of adenosine (m⁶A), is known to be important for the replication of several RNA viruses. Without wishing to be bound by theory, the Inventors believe that the mechanism by which MAT2A inhibitors reduce RNA virus replication may be via inhibition of m⁶A modification of viral RNA due to reduces SAM levels.

Using a further CRISPR/Cas9 screen, the Inventors discovered that TSG101 (Tumor susceptibility gene 101 protein) is also important for BK polyomavirus infection. TSG101 is a component of the endosomal sorting complex required for transport (ESCRT)-1, which mediates intracellular trafficking within the endocytic system. TSG101 has previously been shown to play a role in the envelopment process for various enveloped viruses such as HIV, because the ESCRT complexes are used by such viruses to facilitate their budding through cellular membranes and scission to release the enveloped virions from host cell membranes. Using the non-enveloped DNA virus BKPyV as a model system, the Inventors identified that the expression of the early viral gene large tumour antigen (LTAg) was significantly reduced when expression of TSG101 was disrupted. Advantageously, this suggests that TSG101 also plays an important role in viral entry, even for non-enveloped viruses.

In particular, the Inventors found that proton pump inhibitors, such as prazoles, which function to inhibit TSG101 by targeting the ubiquitin binding domain thereof, significantly reduce viral replication and viral gene expression. By disrupting the normal function of TSG101, prazoles are able to inhibit ESCRT-I-mediated intracellular trafficking.

Advantageously, the Inventors also found that MAT2A inhibitors exhibit increased potency when used in combination with a TSG101 inhibitor.

The invention provides a MAT2A inhibitor and/or a TSG101 inhibitor for use in a method of treating or preventing a viral infection in a subject. The invention also provides a method for treating or preventing a viral infection in a subject, the method comprising administering to the subject a MAT2A inhibitor and/or a TSG101 inhibitor. The invention also provides use of a MAT2A inhibitor and/or a TSG101 inhibitor in the treatment or prevention of a viral infection in a subject.

In some embodiments, the viral infection is caused by a DNA virus. In some embodiments, the DNA virus is a double stranded DNA (dsDNA) virus. dsDNA viruses have a double stranded DNA genome from which mRNA is synthesised by host RNA polymerase. Most dsDNA viruses replicate in the nucleus of the host cell, where they rely upon host cell machinery to replicate the viral genome. In some embodiments, the DNA virus is selected from a polyomavirus, a papillomavirus, an adenovirus, a poxvirus, or a herpesvirus. In some embodiments, the DNA virus is selected from a polyomavirus, a papillomavirus, an adenovirus, or a herpesvirus.

In some embodiments, the DNA virus is a polyomavirus. To date up to 14 polyomaviruses that infect humans have been identified. Polyomaviruses are all thought to have similar replication cycles, and so these viruses would be expected to be susceptible to MAT2A inhibition as demonstrated herein for BKPyV. Polyomaviruses typically cause asymptomatic infections in humans, but have the potential to cause serious diseases and complications, particularly in immunosuppressed subjects. In some embodiments, the subject is immunosuppressed.

In some embodiments, the DNA virus is BKPyV. BKPyV is one of the most prevalent polyomaviruses and is a major cause of PVAN and late-onset haemorrhagic cystitis. In some embodiments, the DNA virus is JC polyomavirus (JCPyV). JCPyV is the most closely related human polyomavirus to BKPyV, and can cause the rare but fatal condition PML. In some embodiments, the DNA virus is Merkel cell polyomavirus (MCPyV).

In some embodiments, the DNA virus is a polyomavirus, and the method comprises treating or preventing polyomavirus associated nephropathy (PVAN). PVAN typically occurs following kidney transplantation and is a known cause of graft loss. In some embodiments, the subject is a kidney transplant patient.

In some embodiments, the DNA virus is a polyomavirus, and the method comprises treating or preventing progressive multifocal leukoencephalopathy (PML). PML occurs almost exclusively in patients with severe immunodeficiency.

In some embodiments, the virus is an RNA virus. In some embodiments, the RNA virus is selected from an alphavirus, an astrovirus, a calicivirus, a flavivirus, and a picornavirus. In some embodiments, the RNA virus is selected from a Sindbis virus, a Semliki Forest virus and Zika virus.

In some embodiments, the viral infection is causes by a non-enveloped virus. In some embodiments, the non-enveloped virus is selected from a polyomavirus, a papillomavirus, an adenovirus, a parvovirus, an astrovirus, a calicivirus, a picornavirus, or a reovirus.

In some embodiments, the invention provides a TSG101 inhibitor for use in a method of treating or preventing a viral infection in a subject, wherein the viral infection is caused by a non-enveloped virus. In some embodiments, the invention provides a TSG101 inhibitor for use in a method of treating or preventing a viral infection in a subject, wherein the viral infection is caused by an enveloped virus.

In some embodiments, the invention provides a MAT2A inhibitor for use in a method of treating or preventing a viral infection in a subject, wherein the viral infection is caused by a non-enveloped virus. In some embodiments, the invention provides a MAT2A inhibitor for use in a method of treating or preventing a viral infection in a subject, wherein the viral infection is caused by an enveloped virus. In some embodiments, the subject is immunosuppressed. In some embodiments, the subject is a transplant patient. In some embodiments, the subject has HIV and/or acquired immune deficiency syndrome (AIDS). In some embodiments, the subject is or has been treated with an immunosuppressive drug, such as natalizumab or rituximab.

In some embodiments, the DNA virus is a polyomavirus, and the method comprises treating or preventing late-onset haemorrhagic cystitis. Late-onset haemorrhagic cystitis may be particularly prevalent in haematopoietic stem cell transplant patients. In some embodiments, the subject is a haematopoietic stem cell transplant patient.

In some embodiments, the DNA virus is a papillomavirus, such as human papillomavirus (HPV). Papillomaviruses are very similar to polyomaviruses in terms of the host cell machinery required for viral genome replication and essential splicing of viral mRNA. Based on the results described herein, papillomaviruses are expected to be susceptible to MAT2A inhibitors and/or TSG101 inhibitors.

In some embodiments, the DNA virus is an adenovirus. Adenovirus infections are common in humans and cause a range of respiratory illnesses ranging from the common cold to pneumonia, croup, and bronchitis. Adenovirus infections can also cause gastroenteritis, cystitis, and conjunctivitis. In some embodiments, the invention provides a MAT2A inhibitor and/or a TSG101 inhibitor for use in a method of treating or preventing a respiratory illness (e.g. the common cold, pneumonia, croup or bronchitis), gastroenteritis, cystitis, or conjunctivitis in a subject. In some embodiments, the invention provides a method for treating or preventing a respiratory illness (e.g. the common cold, pneumonia, croup or bronchitis), gastroenteritis, cystitis, or conjunctivitis in a subject, the method comprising administering to the subject a MAT2A inhibitor and/or a TSG101 inhibitor. In some embodiments, the invention provides use of a MAT2A inhibitor and/or a TSG101 inhibitor in the treatment or prevention of a respiratory illness (e.g. the common cold, pneumonia, croup or bronchitis), gastroenteritis, cystitis, or conjunctivitis in a subject.

In some embodiments, the DNA virus is a poxvirus. Poxviruses are responsible for various diseases, including smallpox and monkeypox. In some embodiments, the invention provides a MAT2A inhibitor and/or a TSG101 inhibitor for use in a method of treating or preventing smallpox or monkeypox in a subject. In some embodiments, the invention provides a method for treating or preventing smallpox or monkeypox in a subject, the method comprising administering to the subject a MAT2A inhibitor and/or a TSG101 inhibitor. In some embodiments, the invention provides use of a MAT2A inhibitor and/or a TSG101 inhibitor in the treatment or prevention of smallpox or monkeypox in a subject.

In some embodiments, the DNA virus is a herpesvirus. Some types of herpesviruses primarily infect humans, including herpes simplex viruses (HSV) 1 and 2 which can cause genital herpes, varicella zoster virus which can cause chickenpox and shingles, and Epstein-Barr virus (EBV) which is a causative agent of various conditions, including glandular fever. In some embodiments, the invention provides a MAT2A inhibitor and/or a TSG101 inhibitor for use in a method of treating or preventing herpes such as genital herpes, chickenpox, shingles or glandular fever in a subject. In some embodiments, the invention provides a method for treating or preventing herpes such as genital herpes, chickenpox, shingles or glandular fever in a subject, the method comprising administering to the subject a MAT2A inhibitor and/or a TSG101 inhibitor. In some embodiments, the invention provides use of a MAT2A inhibitor and/or a TSG101 inhibitor in the treatment or prevention of herpes such as genital herpes, chickenpox, shingles or glandular fever in a subject.

In some embodiments, the viral infection is caused by a nuclear replicating virus. Nuclear replicating viruses rely on various host cell machinery, including splicing machinery. MAT2A inhibition inhibits splicing activity, and so nuclear replicating viruses are expected to be susceptible to MAT2A inhibition. In some embodiments, the nuclear replicating virus is a retrovirus, such as HIV. In some embodiments, the nuclear replicating virus is an influenza virus. In some embodiments, the invention provides a MAT2A inhibitor for use in a method of treating or preventing HIV, AIDS or influenza in a subject. In some embodiments, the invention provides a method for treating or preventing HIV, AIDS or influenza in a subject, the method comprising administering to the subject a MAT2A inhibitor. In some embodiments, the invention provides use of a MAT2A inhibitor in the treatment or prevention of HIV, AIDS or influenza in a subject.

Various types of viruses, including DNA viruses and nuclear replicating viruses, cause asymptomatic infections in most humans. However, these viruses have the potential to cause serious and life threatening infections in subjects who have compromised immune systems. In some embodiments, the subject is immunosuppressed. An immunosuppressed subject is someone who has a weakened immune system and is therefore less able to resist and fight infections. Immunosuppression may be induced by treatment with immunosuppressive drugs that inhibit the subject's immune system. Immunosuppressive drugs are often used to treat autoimmune disorders, and they can also be used in the treatment of cancer. Two exemplary immunosuppressive drugs, natalizumab, which is used to treat multiple sclerosis and Crohn's disease, and rituximab, which is used as an anti-cancer drug, have been associated with an increased risk of PML (1:1,000 and 1:32,000, respectively).

In some embodiments, the subject has been treated or is being treated with an immunosuppressive drug. In some embodiments, the method comprises administering a MAT2A inhibitor and/or a TSG101 inhibitor to the subject prior to administration of an immunosuppressive drug. In some embodiments, the method comprises administering a MAT2A inhibitor and/or a TSG101 inhibitor to the subject concurrently with administration of an immunosuppressive drug. In some embodiments, the method comprises administering a MAT2A inhibitor and/or a TSG101 inhibitor to the subject after administration of an immunosuppressive drug. In some embodiments, the method comprises administering a MAT2A inhibitor and/or a TSG101 inhibitor to the subject for the same period of time as the subject is being treated with the immunosuppressive drug.

Immunosuppressive drugs play an important role in transplantation because they reduce the likelihood of transplant rejection and graft vs host disease. Although there are clear advantages associated with suppressing the immune systems of transplant patients, an unfortunate consequence is that these patients become susceptible to a range of infections, such as viral infections, when they are already medically vulnerable. The immunosuppression required for kidney transplantation and HSC transplantation results in these patients becoming susceptible to viral infections, including infections caused by DNA viruses such as polyomaviruses. As described above, infection by polyomaviruses can have serious and even fatal consequences for these patients.

In some embodiments, the subject is a transplant patient. In some embodiments, the subject is a kidney transplant patient. In some embodiments, the subject is a haematopoietic stem cell (HSC) transplant patient. In some embodiments, the method comprises administering a MAT2A inhibitor and/or a TSG101 inhibitor to the subject prior to transplantation. In some embodiments, the method comprises administering a MAT2A inhibitor and/or a TSG101 inhibitor to the subject after transplantation. In some embodiments, the method comprises administering a MAT2A inhibitor and/or a TSG101 inhibitor to the subject for a defined period of time following transplantation, such as for at least 1 week, at least 2 weeks, at least 3 weeks, at least 4 weeks, at least 2 months, at least 3 months, at least 4 months, at least 5 months, at least 6 months, or at least 1 year following transplantation.

As used herein, a MAT2A inhibitor refers to an agent that inhibits or prevents the activity of MAT2A. MAT2A activity can be inhibited at any level, including transcription, expression, localisation, and cellular activity. In one embodiment, inhibition or prevention of MAT2A activity is associated with decreased transcription of the MAT2A gene, which may be determined by any suitable method in the art, such as qPCR (see below). In one embodiment, inhibition or prevention of MAT2A activity is associated with decreased expression of the MAT2A gene, which may be determined by any suitable method in the art, such as Western blot. In one embodiment, inhibition or prevention of MAT2A activity is associated with altered or loss of MAT2A localisation, which may be determined by any suitable method in the art, such as immunohistochemistry (e.g. immunofluorescence). In one embodiment, inhibition or prevention of MAT2A activity is associated with decreased cellular activity, which may be determined by any suitable method in the art, such as an enzymatic assay of MAT2A activity. In some embodiments, inhibition of MAT2A is identified using a MAT2A inhibitor screening assay (e.g. a commercially-available MAT2A inhibitor screening assay e.g. from BPS Bioscience, California, USA (catalogue number 71402)).

MAT2A inhibitors are known in the art. In some embodiments, the MAT2A inhibitor is:

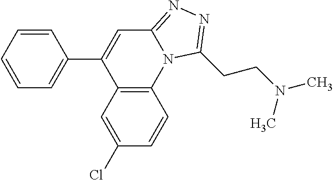 [SMILES: CN(C)CCc1nnc2cc(-c3ccccc3)c3cc(Cl)ccc3n12]

(referred to herein as PF-9366).

In some embodiments, the MAT2A inhibitor is:

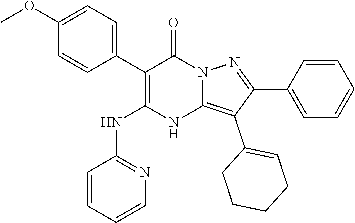 [SMILES: COc1ccc(-c2c(Nc3ccccn3)[nH]c3c(C4=CCCCC4)c(-c4ccccc4)nn3c2=O)cc1]

(referred to herein as AG-270).

In some embodiments, the MAT2A inhibitor is:

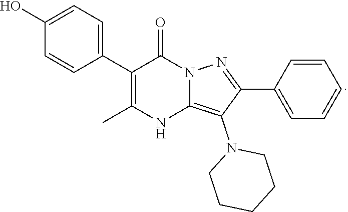 [SMILES: Cc1[nH]c2c(N3CCCCC3)c(-c3ccccc3)nn2c(=O)c1-c1ccc(O)cc1]

In some embodiments, the MAT2A inhibitor is:

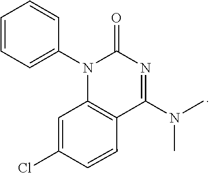 [SMILES: CN(C)c1nc(=O)n(-c2ccccc2)c2cc(Cl)ccc12]

In some embodiments, the MAT2A inhibitor is:

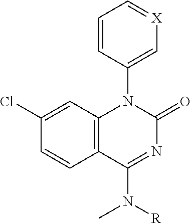

wherein R is Me or Et and X is C or N.

In some embodiments, the MAT2A inhibitor is:

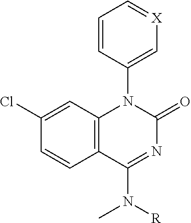

wherein:
(a) R is Me and X is C (referred to herein as AZ28);
(b) R is Me and X is N (referred to herein as AZ31);
(c) R is Et and X is C (referred to herein as AZ32); or
(d) R is Et and X is N (referred to herein as AZ33).

In some embodiments, the MAT2A inhibitor is selected from a MAT2A inhibitor disclosed in WO 2018/045071. In some embodiments, the MAT2A inhibitor is selected from a compound disclosed in Table 1 or Table 2 of WO 2018/045071.

In some embodiments, the MAT2A inhibitor is selected from a MAT2A inhibitor disclosed in WO 2020/139992. In some embodiments, the MAT2A inhibitor is selected from a compound disclosed in Table 1, Table 2, Table 3, or Table 4 of WO 2020/139992.

In some embodiments, the MAT2A inhibitor is selected from a MAT2A inhibitor disclosed in WO 2020/167952. In some embodiments, the MAT2A inhibitor is selected from a compound disclosed in paragraph [0098] or [0115] of WO 2020/167952. In some embodiments, the MAT2A inhibitor is selected from a compound disclosed in Table 1, Table 2, Table 3, Table 4, Table 5, or Table 6 of WO 2020/167952.

In some embodiments, the MAT2A inhibitor is selected from a MAT2A inhibitor disclosed in WO 2020/123395. In some embodiments, the MAT2A inhibitor is selected from a compound disclosed in Table 1 or Table 2 of WO 2020/123395.

In some embodiments, the MAT2A inhibitor is selected from a MAT2A inhibitor disclosed in WO 2021/252678. In some embodiments, the MAT2A inhibitor is selected from a compound disclosed in Table 1 of WO 2021/252678.

In some embodiments, the MAT2A inhibitor is selected from a MAT2A inhibitor disclosed in WO 2021/158792. In some embodiments, the MAT2A inhibitor is selected from a compound disclosed in paragraph [00131], or of WO 2021/158792.

In some embodiments, the MAT2A inhibitor is a small molecule. As used herein, small molecules are low molecular weight compounds, typically organic compounds with a maximum molecular weight of 900 Da, allowing for rapid diffusion across cell membranes. Libraries of small molecules can be tested for their ability to inhibit or prevent MAT2A activity using methods described in the art, see e.g. WO 2018/045071, Example 2 therein.

In some embodiments, the MAT2A inhibitor is selected from a peptide, a cyclic peptide, a polypeptide, a peptidomimetic, and a protein.

In some embodiments, the MAT2A inhibitor is an oligonucleotide that represses MAT2A gene expression, e.g. by binding to MAT2A DNA or mRNA. In some embodiments, the MAT2A inhibitor is an antisense oligonucleotide, shRNA, siRNA, microRNA, or an aptamer. Aptamers are generally nucleic acid molecules that bind a specific target molecule. Aptamers can be engineered in vitro, are readily produced by chemical synthesis, possess desirable storage properties, and elicit little or no immunogenicity in therapeutic applications. As used herein, “aptamer” refers in general to a single or double stranded oligonucleotide or a mixture of such oligonucleotides, wherein the oligonucleotide or mixture is capable of binding specifically to a target.

In some embodiments, the MAT2A inhibitor is an antibody. As used herein, the term antibody encompasses the use of a monoclonal antibody or polyclonal antibody, as well as the antigen-binding fragments of a monoclonal or polyclonal antibody, or a peptide which binds to MAT2A with specificity. The antibody may be a Fab, F(ab′)2, Fv, scFv, Fd or dAb.

As used herein, a TSG101 inhibitor refers to an agent that inhibits or prevents the activity of TSG101. TSG101 activity can be inhibited at any level, including transcription, expression, localisation, and cellular activity. In one embodiment, inhibition or prevention of TSG101 activity is associated with decreased transcription of the TSG101 gene, which may be determined by any suitable method in the art, such as qPCR (see below). In one embodiment, inhibition or prevention of TSG101 activity is associated with decreased expression of the TSG101 gene, which may be determined by any suitable method in the art, such as western blot. In one embodiment, inhibition or prevention of TSG101 activity is associated with decreased cellular activity, which may be determined by any suitable method in the art, such as an enzymatic assay of TSG101 activity.

In some embodiments, the TSG101 inhibitor is selected from a small molecule, a peptide, a cyclic peptide, and a peptidomimetic.

In some embodiments, the TSG101 inhibitor is a prazole. In some embodiments, the TSG101 inhibitor is selected from Ilaprazole, omeprazole, esomeprazole, dexlansoprazole, lansoprazole, pantoprazole, rabeprazole, and tenatoprazole.

In some embodiments, the TSG101 inhibitor is selected from an oligonucleotide, optionally wherein the TSG101 inhibitor is an antisense oligonucleotide, a shRNA, a siRNA, a microRNA, or an aptamer.

In some embodiments, the TSG101 inhibitor is an antibody.

The therapeutic use or method of the invention may comprise administering a therapeutically effective amount of a MAT2A inhibitor and/or a TSG101 inhibitor, either alone or in combination with one or more additional therapeutic agent(s) or treatment(s). When used in combination with one or more additional therapeutic agent(s) or treatment(s), the MAT2A inhibitor and/or the TSG101 inhibitor may be administered before, simultaneously with, or after the administration of the one or more additional therapeutic agent(s) or treatment(s). In some embodiments, the therapeutic use or method of the invention comprises administering a MAT2A inhibitor and a TSG101 inhibitor simultaneously. In some embodiments, the methods described herein comprise administering a MAT2A inhibitor and a TSG101 inhibitor concurrently, e.g. administering the MAT2A inhibitor and then the TSG101 inhibitor, or administering the TSG101 inhibitor and then the MAT2A inhibitor.

As used herein, the term “treatment” or “treating” embraces therapeutic measures. Treatment of a viral infection can be characterised by a reduction in viral load and/or a reduction in disease symptoms.

A MAT2A inhibitor and/or a TSG101 inhibitor may be used as a preventative therapy. As used herein, the term “preventing” or the like includes preventing the onset of symptoms associated with viral infection. The term “preventing” includes preventing a viral infection from occurring in a subject that may be susceptible to the infection and also preventing the occurrence of symptoms associated with a viral infection.

As used herein, the term “subject” may refer to an animal including, but not limited to, members of the human, primate, equine, porcine, bovine, murine, rattus, canine and feline species. In some embodiments, the subject is a mammal. The subject is typically a human. As used herein, the term “patient” may be used interchangeably with “subject”. The subject may have been diagnosed with having a viral infection using known clinical methods. The subject may be asymptomatic.

A MAT2A inhibitor and/or a TSG101 inhibitor may be linked (covalently or non-covalently) to a targeting ligand designed to facilitate the uptake into the cell. The targeting ligand may comprise a compound that recognises a cell, tissue or organ specific element facilitating cellular uptake and/or a compound that facilitates uptake into cells.

A MAT2A inhibitor and/or a TSG101 inhibitor can be combined or administered in addition to a pharmaceutically acceptable carrier, diluent and/or excipient. Alternatively or in addition the MAT2A inhibitor and/or the TSG101 inhibitor can further be combined with one or more of a salt, excipient, diluent, immunoregulatory agent, antiviral agent and/or antimicrobial compound.

Generally, the carrier is a pharmaceutically-acceptable carrier. Non-limiting examples of pharmaceutically acceptable carriers include water, saline, and phosphate-buffered saline. In some embodiments, the MAT2A inhibitor and/or the TSG101 inhibitor may be part of a composition in lyophilized form, in which case the composition may include a stabilizer, such as BSA. In some embodiments, it may be desirable to formulate the composition with a preservative, such as thiomersal or sodium azide, to facilitate long term storage. Examples of buffering agents include, but are not limited to, sodium succinate (pH 6.5), and phosphate buffered saline (PBS; pH 6.5 and 7.5).

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administration of the MAT2A inhibitor and/or the TSG101 inhibitor is generally by conventional routes e.g. intravenous, subcutaneous, intraperitoneal, or mucosal routes. The administration may be by parenteral injection, for example, a subcutaneous, intradermal or intramuscular injection. For example, formulations comprising a MAT2A inhibitor and/or a TSG101 inhibitor may be particularly suited to administration intravenously, intramuscularly, intradermally, or subcutaneously. Administration of a small molecule MAT2A inhibitor or TSG101 inhibitor may be by injection, such as intravenously, intramuscularly, intradermally, or subcutaneously, or by oral administration.

Accordingly, the MAT2A inhibitor and/or the TSG101 inhibitor may be formulated in an injectable form, either as liquid solutions or suspensions. Solid forms suitable for solution in, or suspension in, liquid prior to injection may alternatively be prepared.

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

The dosage ranges for administration of the MAT2A inhibitor and/or the TSG101 inhibitor are those which produce the desired therapeutic effect. It will be appreciated that the dosage range required depends on the precise nature of the compound, the route of administration, the nature of the formulation, the age of the patient, the nature, extent or severity of the patient's condition, contraindications, if any, and the judgement of the attending physician. Variations in these dosage levels can be adjusted using standard empirical routines for optimisation. Similarly, the dose of MAT2A inhibitor and/or TSG101 inhibitor can be readily determined by one of skill in the art, and is a dose that inhibits virus replication and/or gene expression, e.g. DNA virus replication and/or gene expression.

It will be appreciated that reference to “and/or” embraces either option alone, or in combination. For example, reference to “MAT2A inhibitor and/or TSG101 inhibitor” (and the like) embraces MAT2A inhibitor; TSG101 inhibitor; and MAT2A inhibitor and TSG101 inhibitor. Thus, in some embodiments, “MAT2A inhibitor and/or TSG101 inhibitor” embraces MAT2A inhibitor and optionally TSG101 inhibitor. In some embodiments, “MAT2A inhibitor and/or TSG101 inhibitor” embraces TSG101 inhibitor and optionally MAT2A inhibitor. In some embodiments, “MAT2A inhibitor and/or TSG101 inhibitor” embraces TSG101 inhibitor and MAT2A inhibitor.

### EXAMPLES

The invention will be further clarified by the following examples, which are intended to be purely exemplary of the invention and are in no way limiting.

### Example 1

A genome-wide CRISPR screen was designed to identify genes important for BK Polyomavirus (BKPyV) entry and early gene expression in human telomerase reverse transcriptase (hTERT) immortalised, Cas9 expressing, Renal Proximal Tubule (Cas9-RPTE/TERT1) cells.

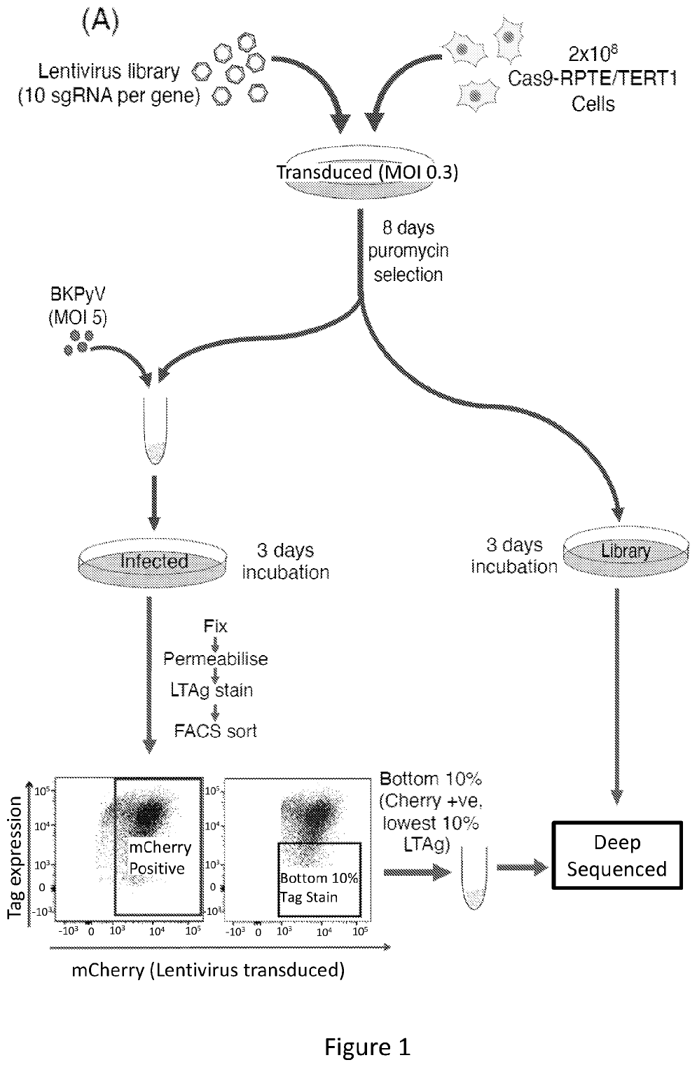

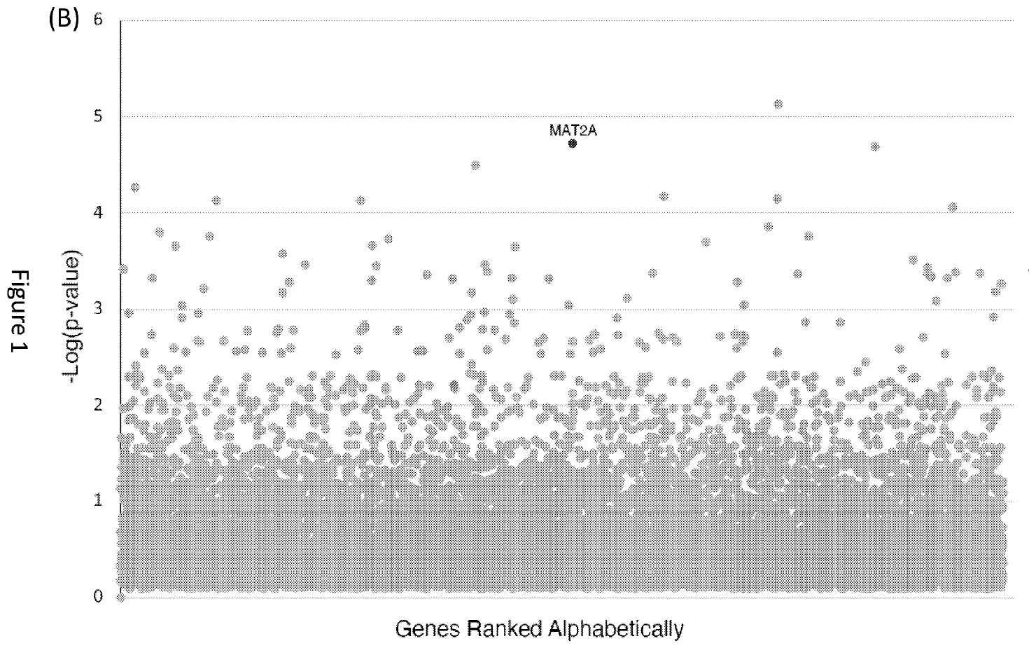

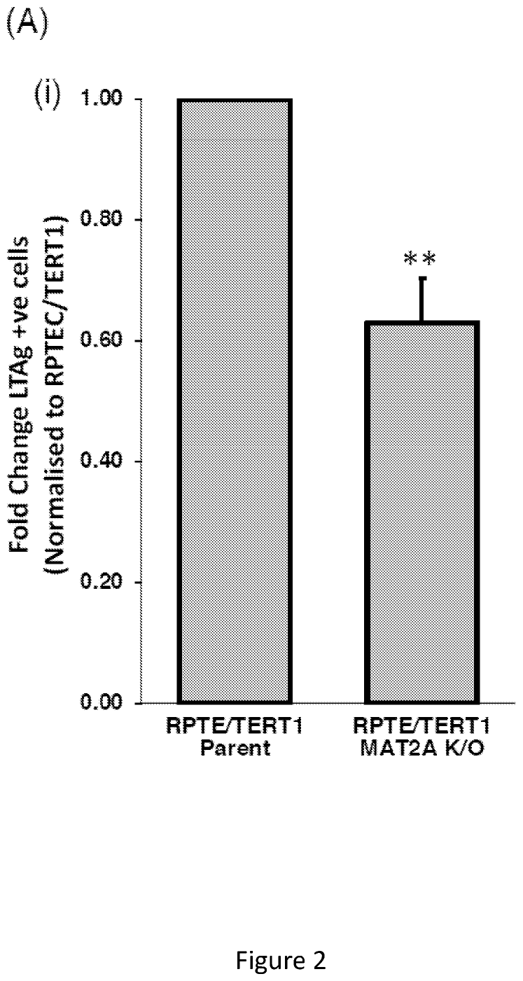

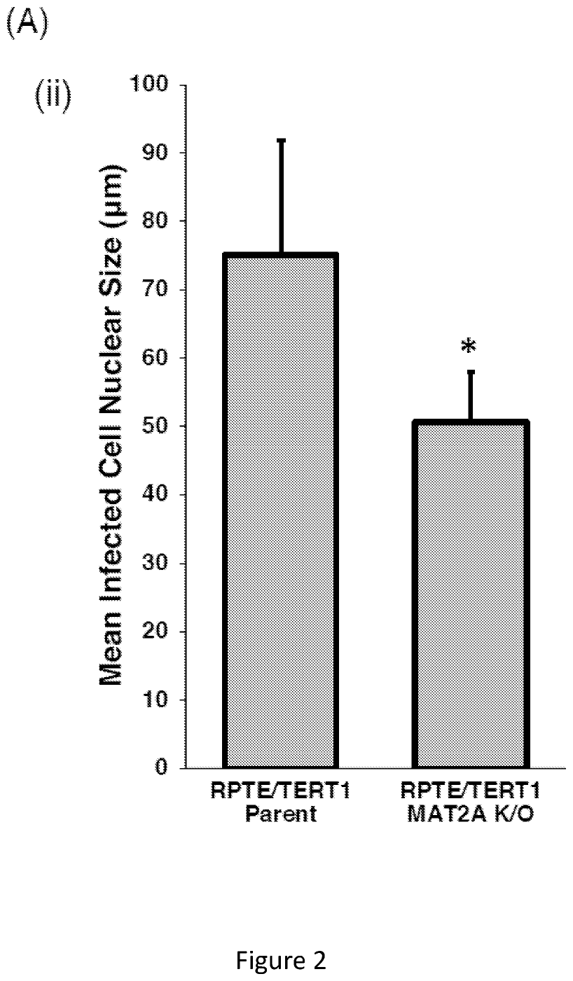

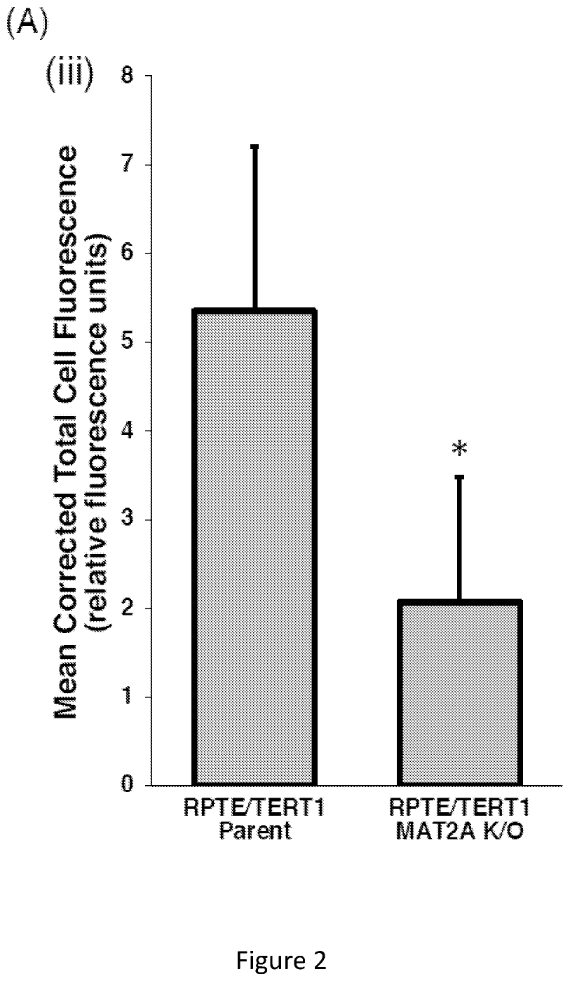

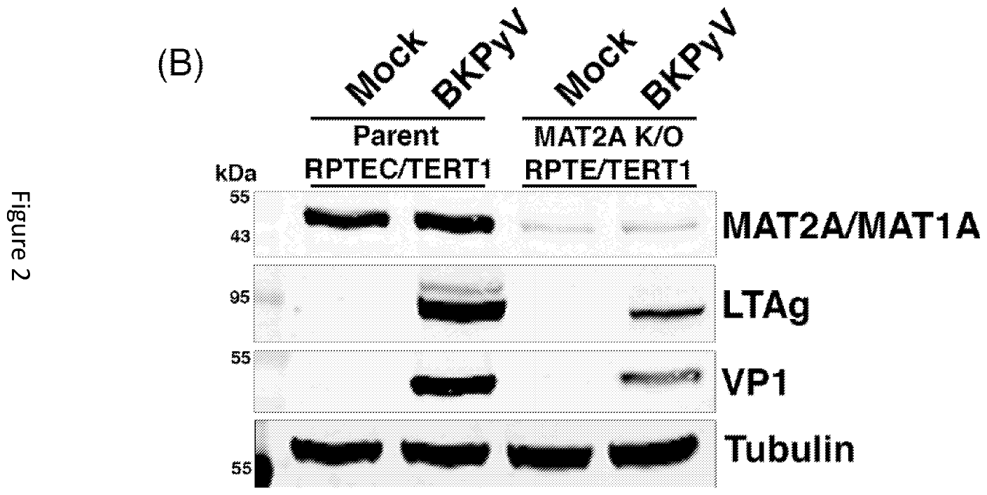

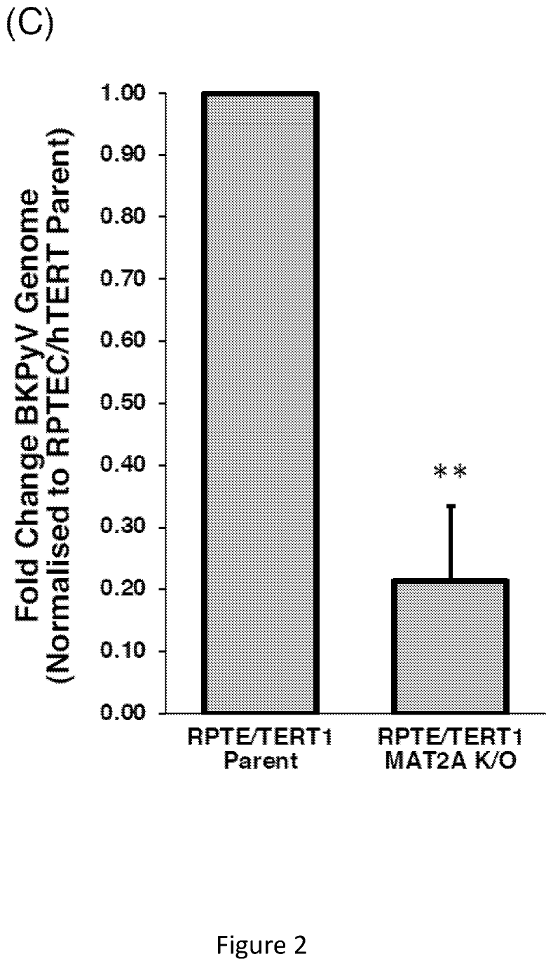

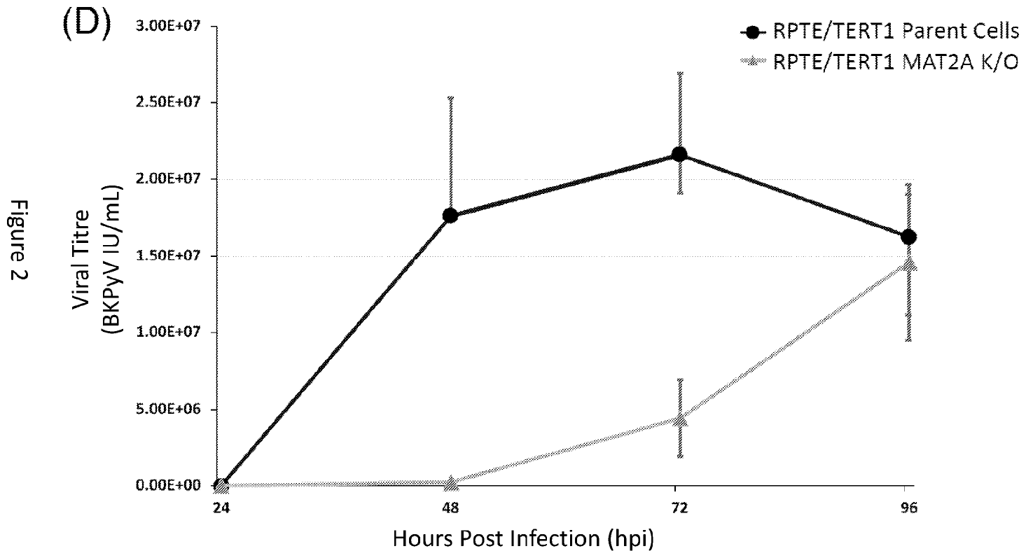

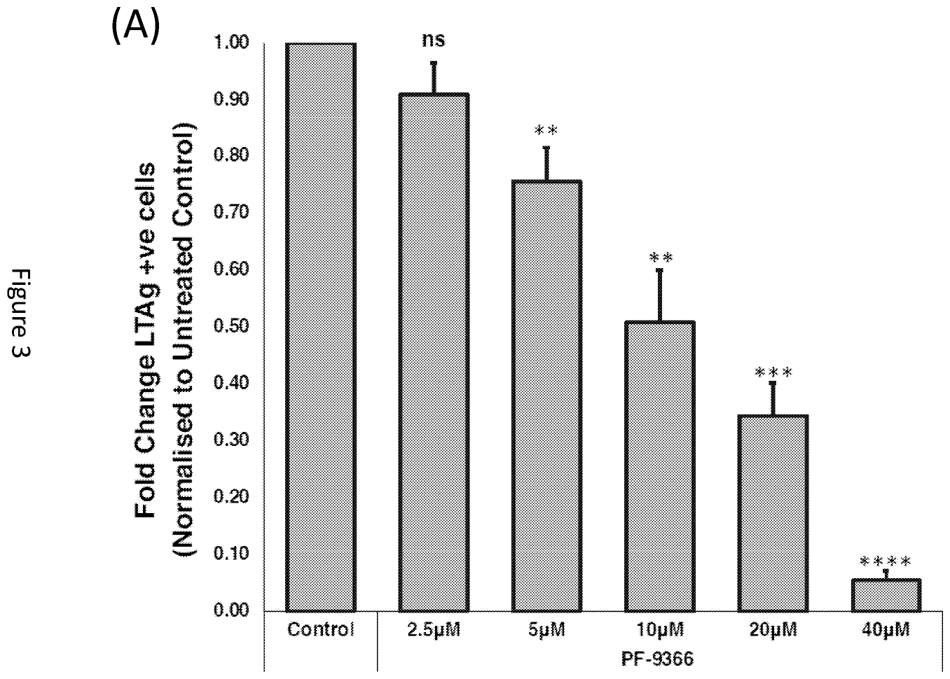

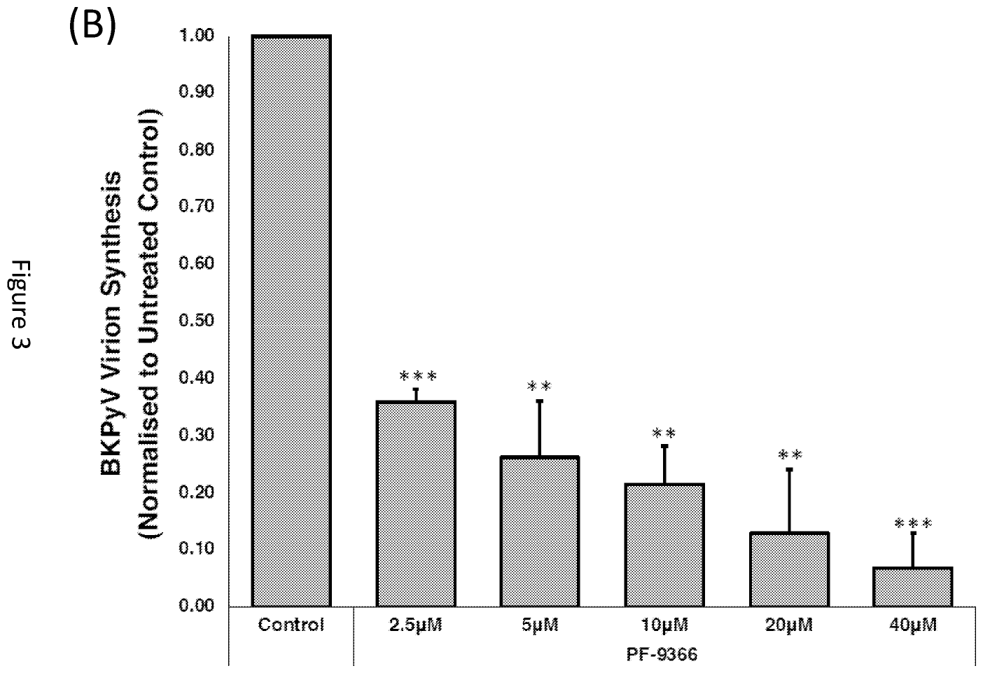

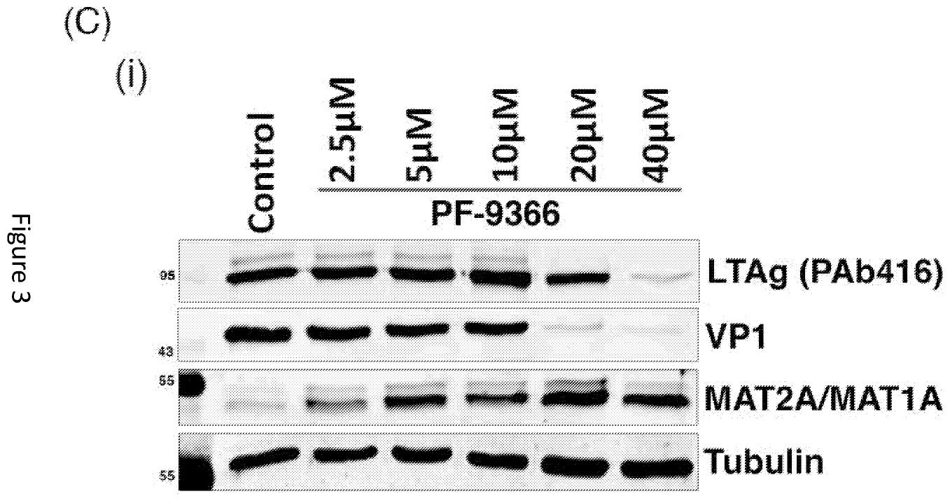

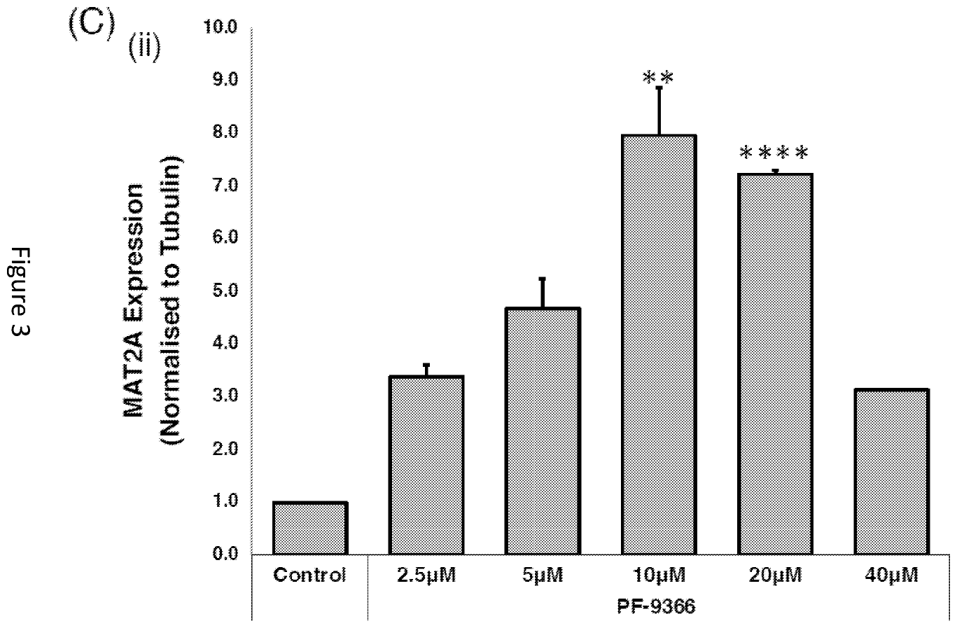

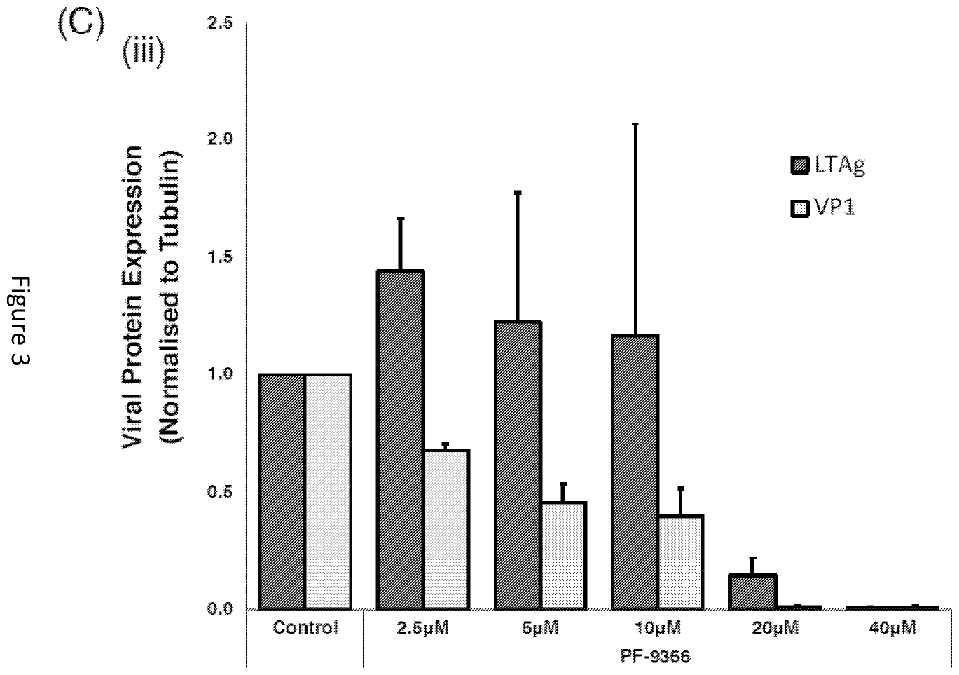

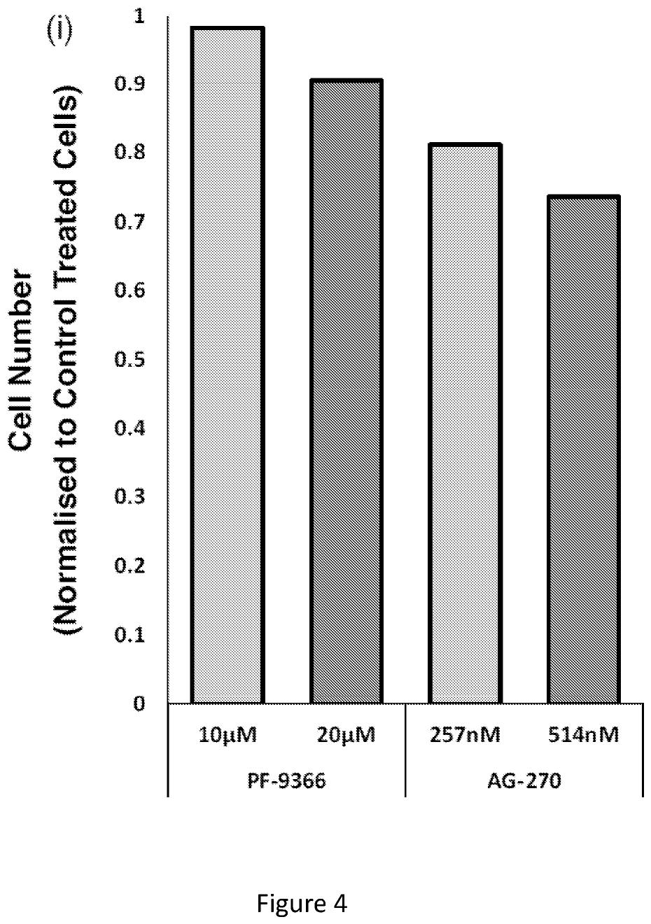

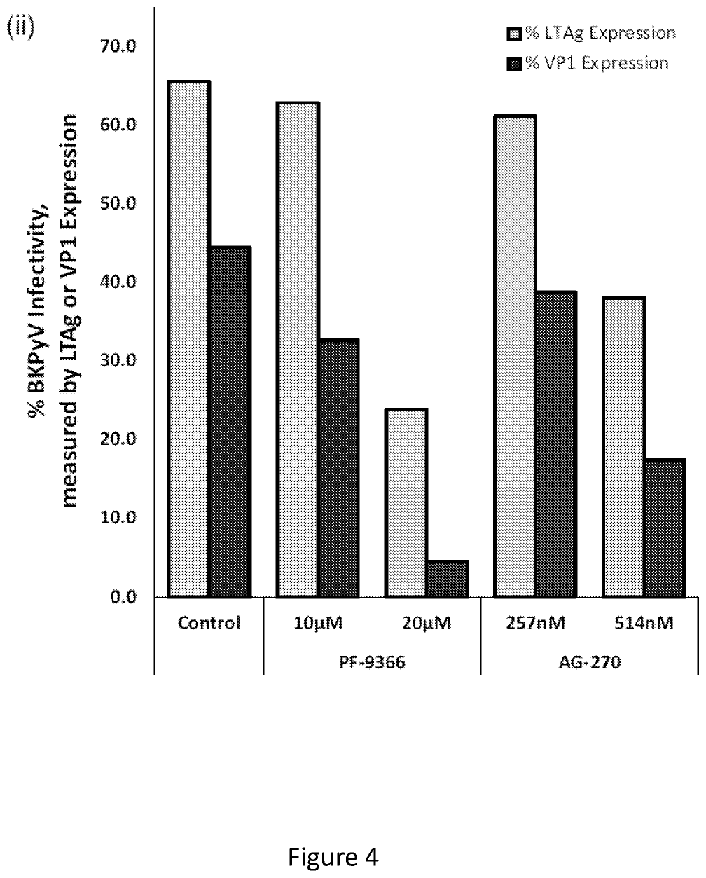

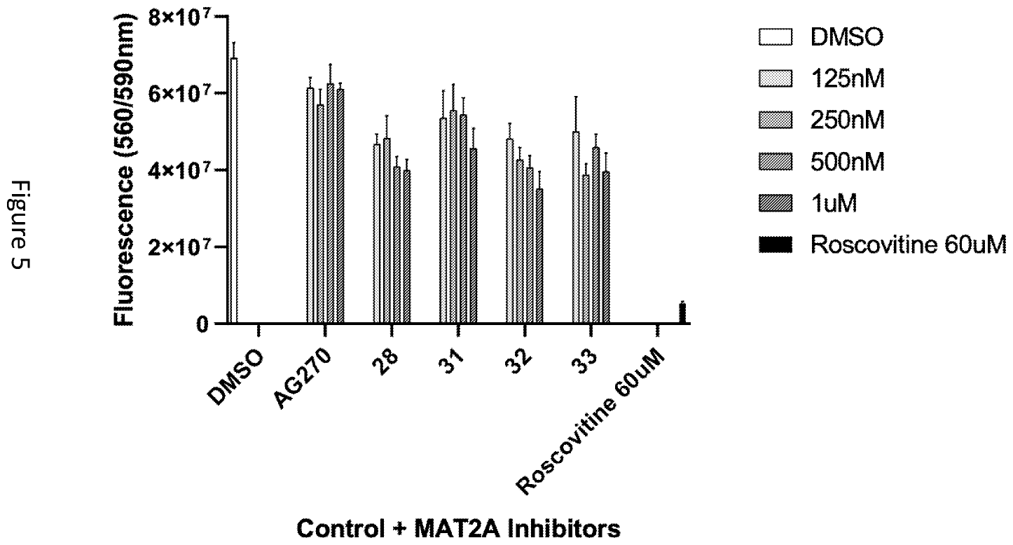

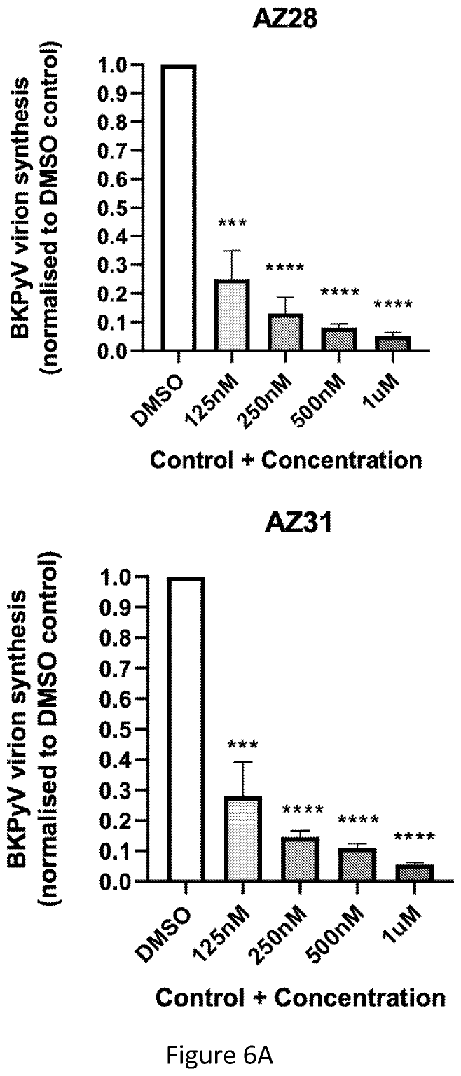

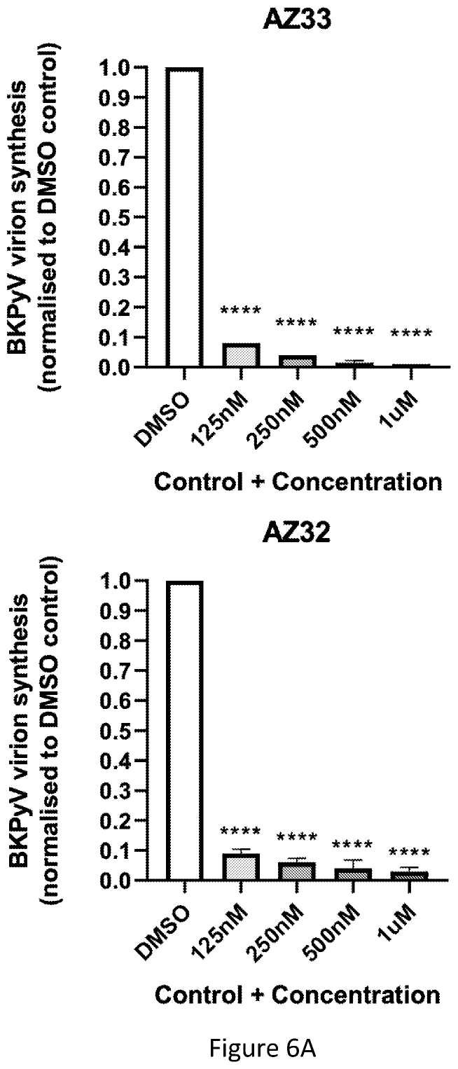

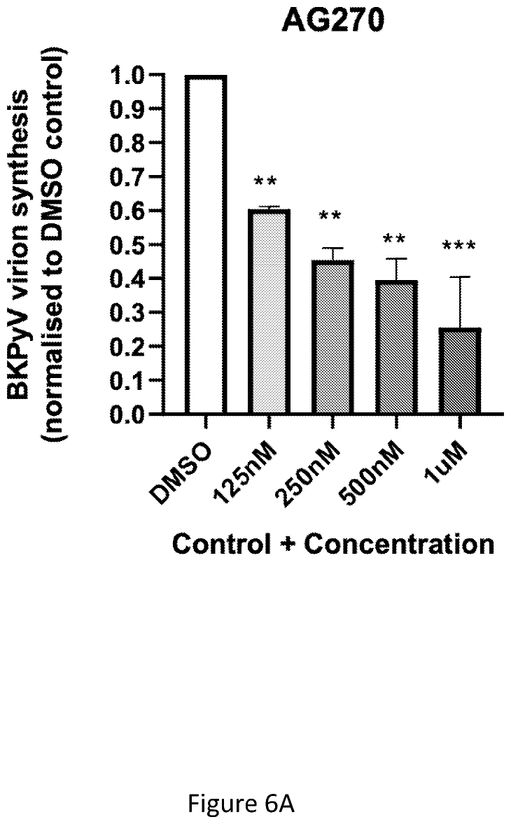

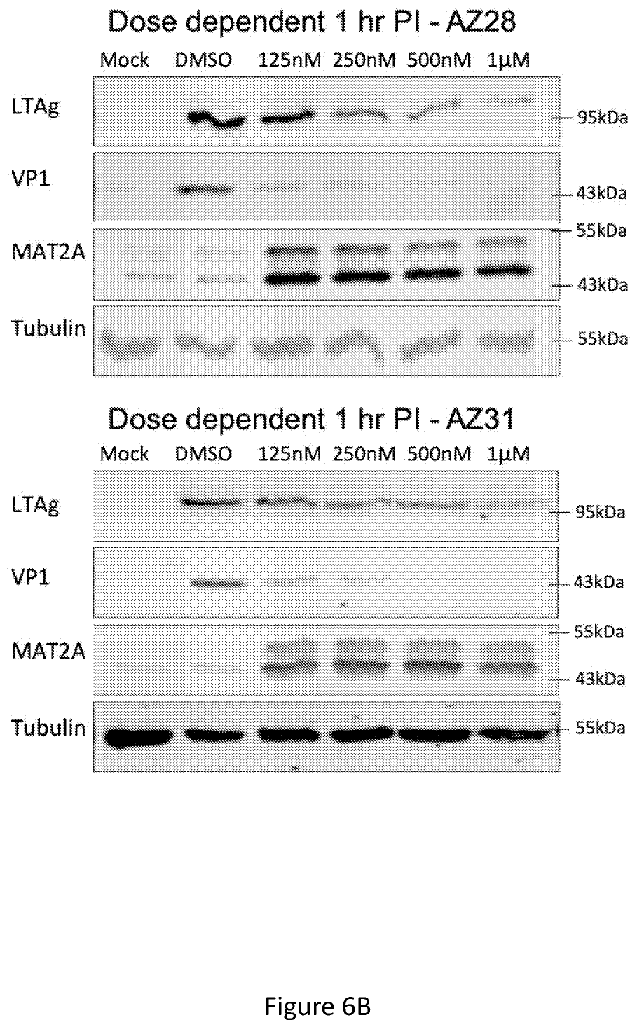

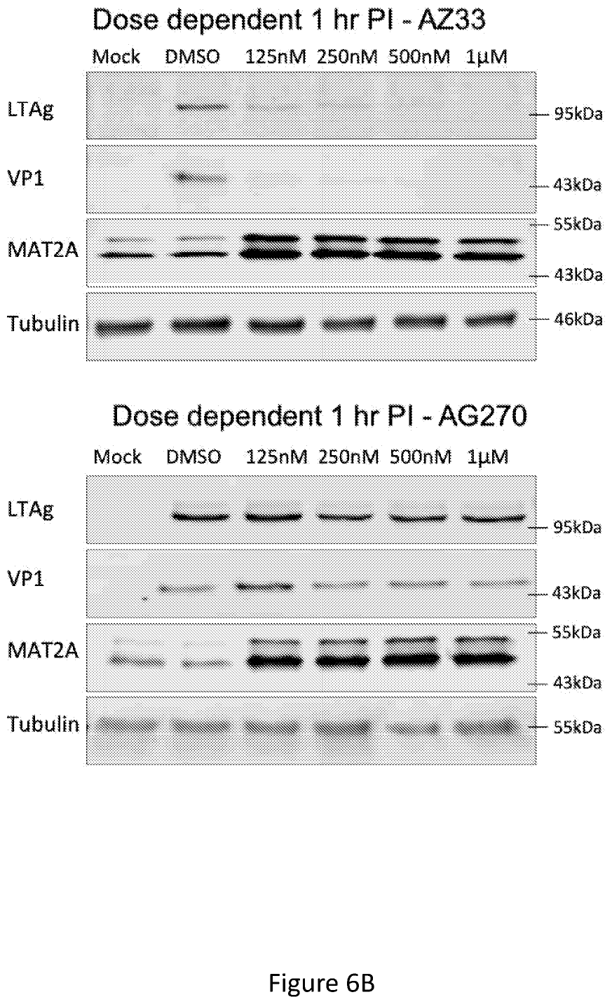

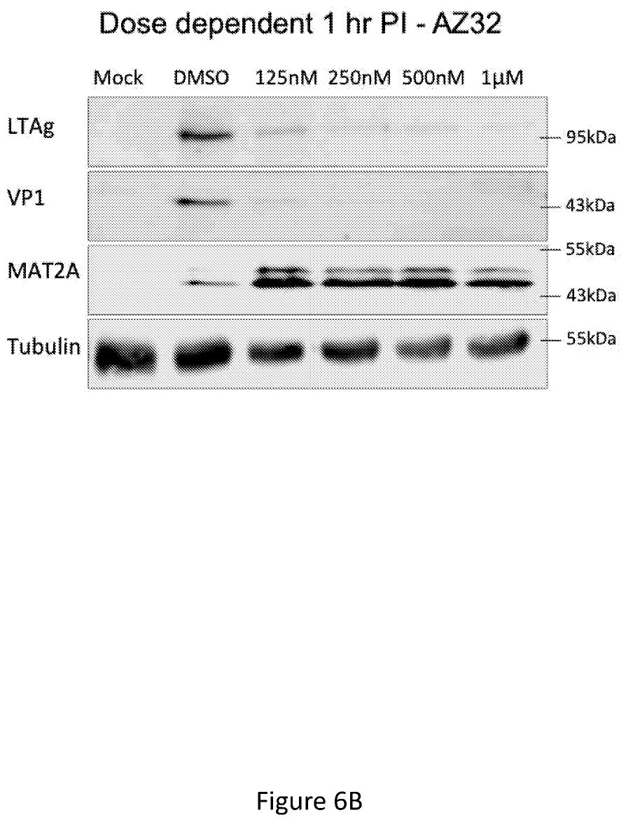

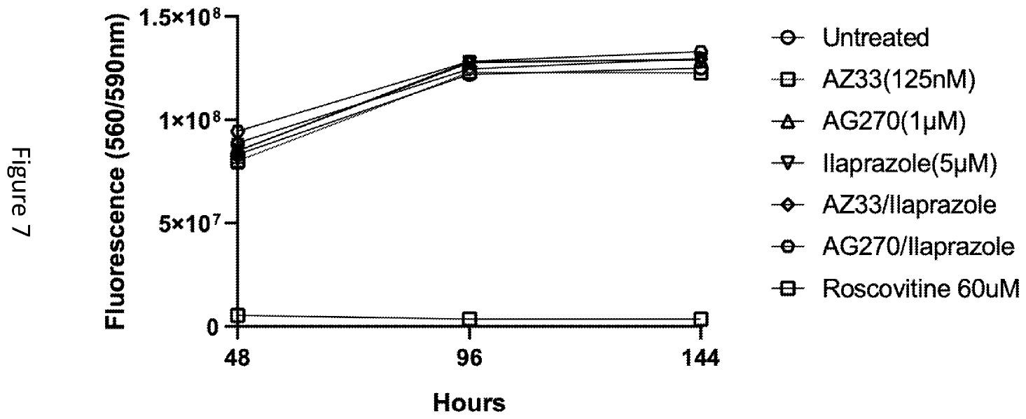

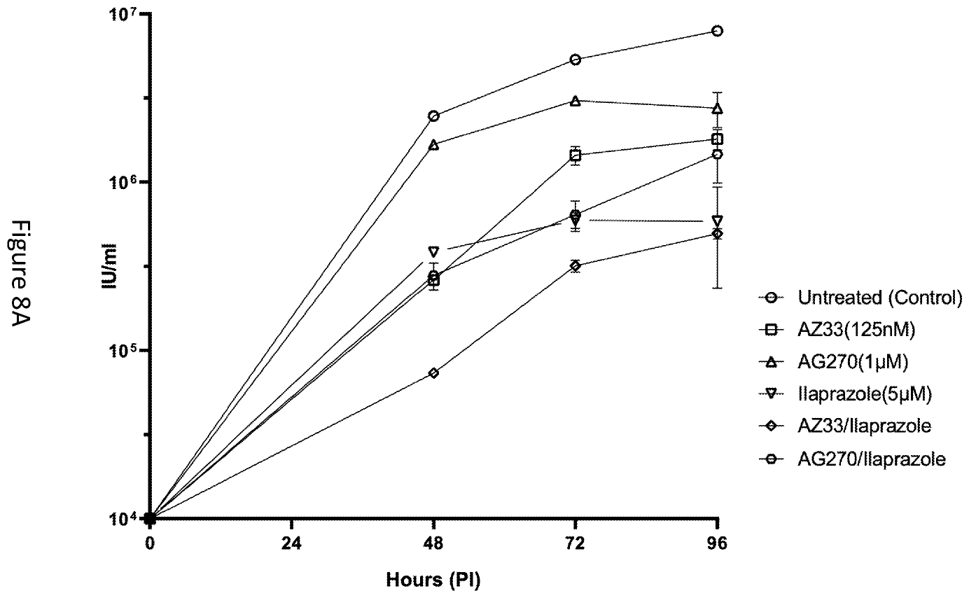

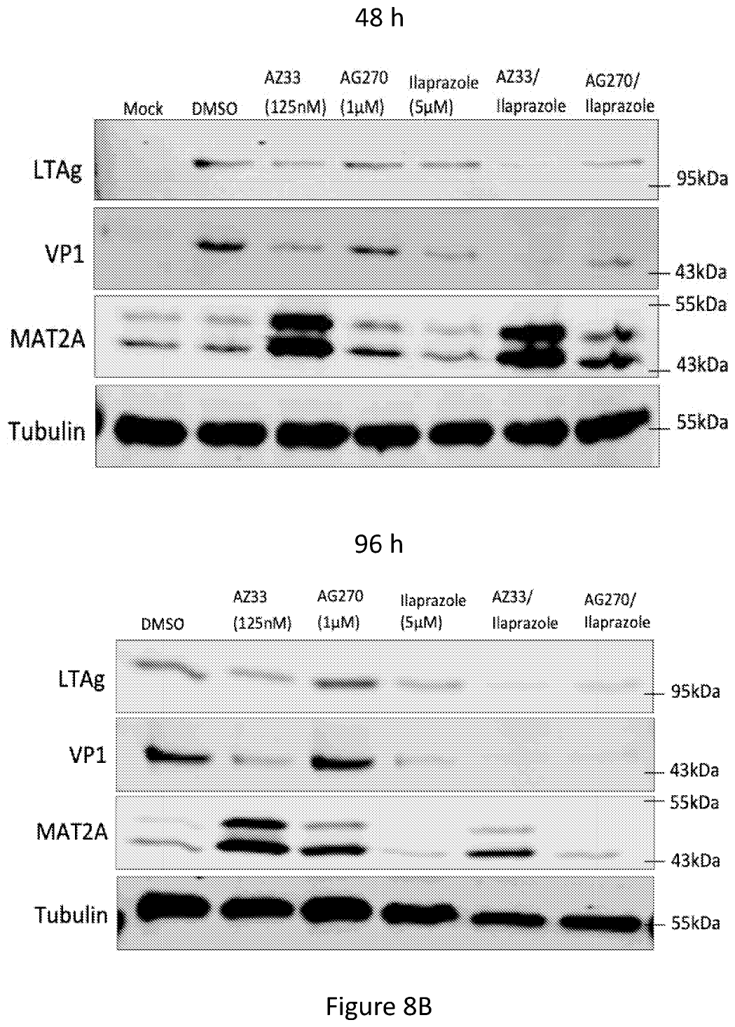

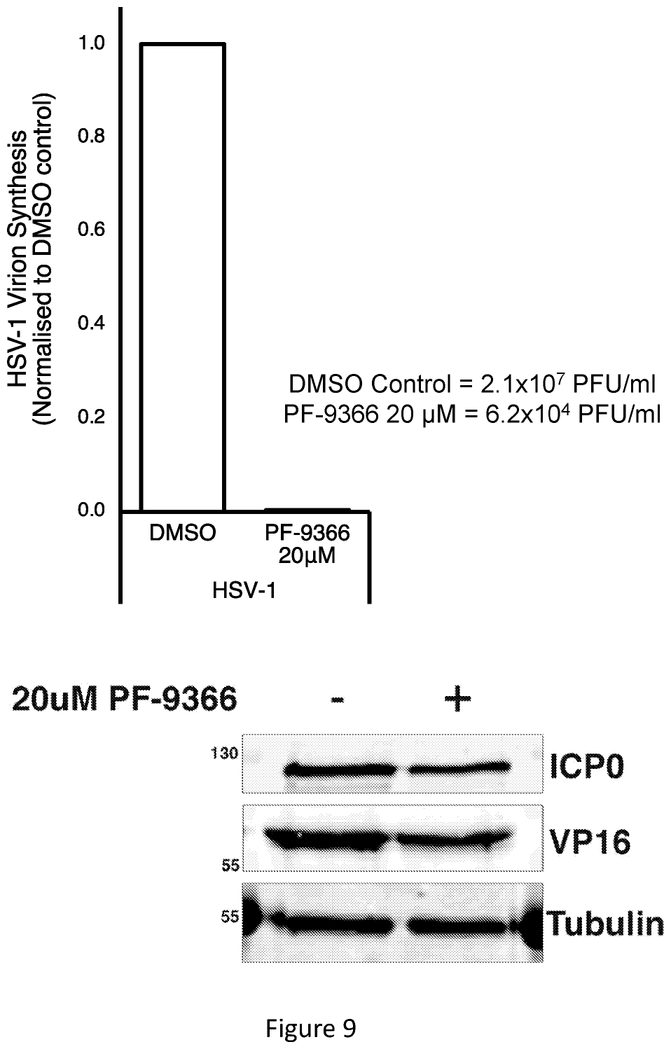

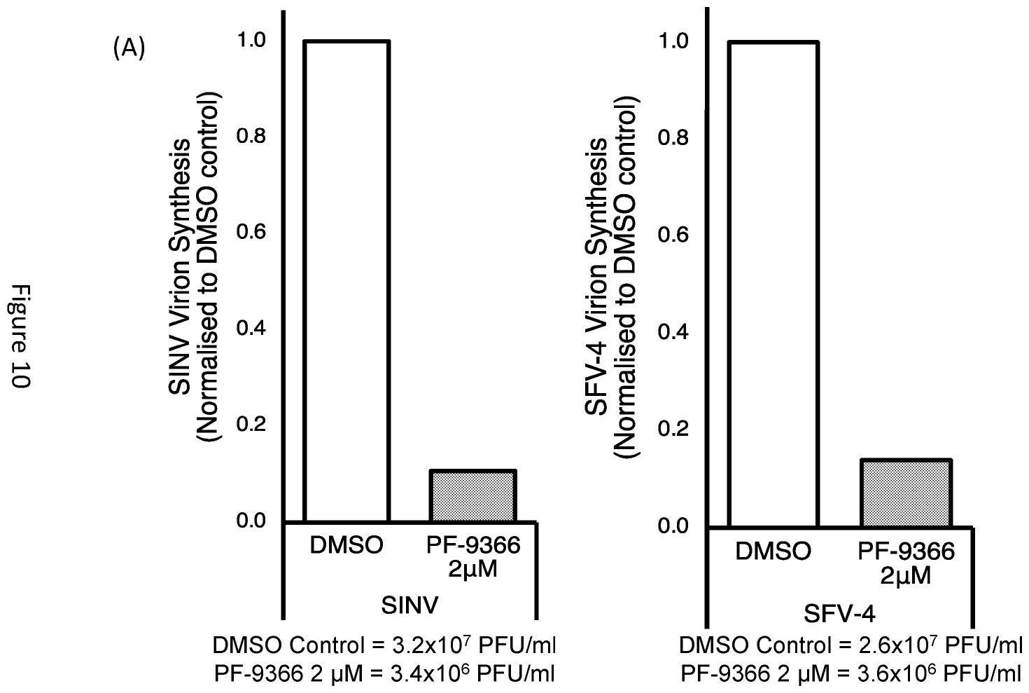

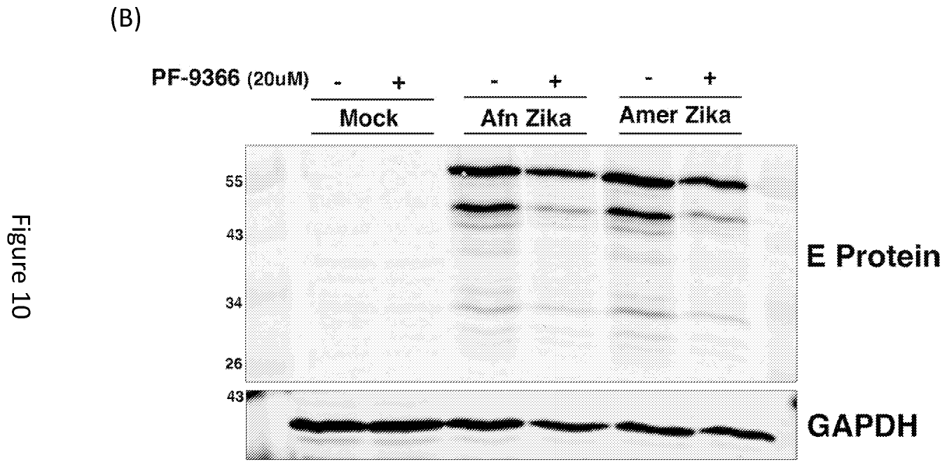

The ˜20,500 human genes were each targeted by 10 unique sgRNAs in an mCherry/Puromycin lentiviral vector. Once transduced and puromycin selected for 8 days, cells were either infected with BKPyV at a multiplicity of infection (MOI) of 5 infectious units per cell or kept as a reference library. At 72 hours post infection (hpi) infected cells were fixed and immuno-stained for the early gene product Large T Antigen (LTAg). Cells were then sorted by fluorescence-activated cell sorting (FACS), firstly for expression of mCherry, indicative of successful sgRNA transduction, and then secondly for lowest 10% of cells based on LTAg signal. This strategy enriches for sgRNA transduced cells where BK polyomavirus infection is inhibited. The relative abundance of sgRNA sequences within the sorted population of mCherry positive but low LTAg expressing cells was then determined by next-generation sequencing (NGS) of the sorted population comparing directly to the unsorted library (FIG. 1A).

The over-representation of genes for which sgRNA sequences were enriched in the sorted proportion of cells compared to the library was then characterised in a dot-plot, where each gene was plotted against the −Log (p-value) (FIG. 1B). One of the most significantly enriched genes was MAT2A, identified by 9 of the 10 sgRNAs.

The MAT2A gene encodes for the protein S-adenosylmethionine synthase isoform type-2 (MAT2A) protein, an enzyme which catalyses S-adenosylmethionine (SAM) formation from methionine and ATP. SAM is an essential factor for methylation reactions which are involved in various cellular functions including RNA metabolism, gene expression (transcription and translation) and genome replication.

To validate the importance of MAT2A for BKPyV infection, a MAT2A gene knockout cell line was generated by CRISPR/Cas9 gene editing (RPTE/TERT1 MAT2A K/O cells) followed by single-cell cloning, and successful knockout or gene function was confirmed by sequencing to identify the INDELS present in both copies of the MAT2A gene. One MAT2A gene copy contains a single base insertion leading to a frameshift and premature stop codon in place of codon 57, and the other MAT2A gene copy contains a single base substitution mutating a conserved alanine at residue 55 (A55) within the enzyme active site. The RPTE/TERT1 MAT2A K/O or parental RPTE/TERT1 cell line were infected with BKPyV (MOI 3). At 48 hpi cells were fixed in 3% formaldehyde and immunofluorescence stained for LTAg expression. The number of LTAg positive cells were counted, normalised to the parental line (FIG. 2A(i)). A ˜40% reduction in the number of LTAg positive cells was observed in RPTE/TERT1 MAT2A K/O cells when compared to the parental cell line (p<0.01, n=3). Furthermore, the nuclear size of each LTAg positive cell, an indicator of viral genome replication, was significantly reduced in RPTE/TERT1 MAT2A K/O cells (FIG. 2A(ii), p<0.05), whilst the total fluorescence of each LTAg positive cell was ˜60% lower in RPTE/TERT1 MAT2A K/O cells compared to parental RPTE/TERT1 cells (FIG. 2A(iii), p<0.05). These data demonstrate that loss of MAT2A expression inhibits the ability of BKPyV to establish early viral gene expression.

A reduction in nuclear size in MAT2A K/O cells suggests that aspects of BKPyV replication beyond early gene expression may be affected by MAT2A knockout. To investigate this RPTE/TERT1 MAT2A K/O and parental RPTE/TERT1 cells were infected with BKPyV (MOI 3) or mock infected, cell pellets were harvested at 48 hpi, and protein expression levels were analysed by Western blotting. These data revealed that expression of both early (LTAg) and late (VP1) viral genes were substantially reduced in MAT2A knockout cells (FIG. 2B). Western blot analysis also revealed a dramatically reduced signal for MAT2A protein expression. The residual signal is likely to be the MAT1A homolog, which is recognised by the available antibody due to high level of sequence similarity (shown in FIG. 2B as a faint band), although a low level of expression of destabilised A55 mutant MAT2A cannot be ruled out.

To investigate whether BKPyV genome replication was also affected by MAT2A knockout RPTE/TERT1 MAT2A K/O and parental RPTE/TERT1 cells were infected with BKPyV (MOI 3), cell pellets were harvested at 48 hpi and viral DNA levels were analysed by qPCR, and normalised to cellular GAPDH gene as a control for cell number. This revealed MAT2A knockout had a greater effect on genome replication, exhibiting an ˜80% reduction in genome numbers in cells lacking MAT2A expression (FIG. 2C, p<0.05, n=3).

To investigate the effects on production of infectious virus, RPTE/TERT1 MAT2A K/O cells and parental RPTE/TERT1 cells were infected with BKPyV (MOI 5) and harvested every 24 hrs until 96 hpi. The level of total infectious virus in each sample was determined by fluorescence focus unit (FFU) assays (FIG. 2D). These data demonstrated a substantial inhibition of BKPyV replication in cells lacking MAT2A expression.

To further investigate whether MAT2A inhibition, rather than gene knockout, could also inhibit BKPyV replication parental RPTE/TERT1 cells were treated with the MAT2A inhibitor PF-9366 at a range of concentrations, or DMSO treated as a control. Treatment began 3 hours prior to infection and continued throughout infection. Cells were infected with BKPyV (MOI 1), at 1 hpi cells were washed with PBS and replenished with fresh drug treated media. At 48 hpi cells were fixed with 3% formaldehyde and stained for LTAg expression, and the nuclei counterstained with DAPI. The number of LTAg positive cells were counted and normalised to the total cell number in each condition, and the fold change in proportion of LTAg positive cells for each concentration of PF-9366 was normalised to untreated control. At PF-9366 concentrations of 5 μM and above the number of LTAg positive cells were significantly reduced, and up to ˜95% reduction in LTAg positive cells was observed at 40 μM (FIG. 3A, p<0.0001, n=3).

The ability for PF-9366 to inhibit the production of infectious virus was then investigated. RPTE/TERT1 cells were once again treated with a concentration range of PF-9366 or DMSO as a control prior to infection, and as before, infected with BKPyV (MOI 1) then washed and media replaced at 1 hpi. At 48 hpi total virus was harvested from each condition and an FFU assay was conducted to determine BKPyV virus titre (FIG. 3B). As noted with MAT2A gene knockout, the effect of MAT2A inhibition on virus synthesis was much more profound than the effects on LTAg expression. Cells treated with the lowest concentration (2.5 μM) of PF-9366 demonstrated a significant 60% reduction in infectious virus production (p<0.001, n=3), and at the maximum 40 μM PF-9366 treatment infectious virus production was reduced by ˜95% (p<0.001, n=3).

It has previously been noted by Quinlan et al. (Nat Chem Biol. 2017 13 (7): 785-792) that treatment with PF-9366 can increase MAT2A expression as a compensatory mechanism within cells. To investigate this, cells were once again treated with a range of PF-9366 for 3 hours prior to infection (or DMSO treated). Cells were infected with BKPyV (MOI 3), washed at 1 hpi, and fresh drug treated media was added. At 48 hpi cells were harvested and protein expression was analysed by western blotting (FIG. 3C(i)). MAT2A expression was quantified from band intensities, normalised to the tubulin loading control and plotted (FIG. 3C(ii), n=3). A clear increase in the cellular expression of MAT2A was observed, up to an 8-fold increase at 10 μM PF-9366. Western blot analysis was also conducted on early viral gene (LTAg) and late viral gene (VP1) expression (FIG. 3C(iii), n=3). Reduced expression of LTAg was observed at the two highest concentrations of PF-9366 whereas VP1 expression showed a dose-dependent reduction across the concentration range of PF-9366 used (FIG. 3C).

To investigate whether MAT2A inhibition by a structurally different inhibitor to PF-9366 could cause similar effects, the effect of AG-270 on BKPyV infection was investigated. As above, drugs were used to treat cells from 3 hours prior to infection. Drug concentrations used were the IC50 value defined by inhibition of cell proliferation (proliferative IC50) or double the proliferative IC50, and DMSO was used as a control. Cells were infected with BKPyV (MOI 1), at 1 hpi cells were washed and fresh drug-containing media was added. At 48 hpi cells were fixed in 3% formaldehyde and stained for DAPI, viral early gene LTAg, and viral late gene VP1. The number of cells in each condition were counted and normalised to DMSO treated cells (FIG. 4A(i)). The number of cells represents both cell viability, and the ability of the drug in question to inhibit cellular proliferation. Both PF-9366 and AG-270, at both concentrations, showed cells to be greater than 70% viable. Furthermore, when LTAg and VP1 positive cells were counted, as a proportion of DAPI-positive cells (FIG. 4A(ii)) both inhibitors showed a reduced early gene (LTAg) and late gene (VP1) expression, particularly at the doses representing double proliferative IC50 (20 UM PF-9366, 514 nM AG-270).

Taken together, these data clearly demonstrate that MAT2A activity is important for BKPyV replication, and inhibition of MAT2A function by gene knockout and two independent MAT2A inhibitors significantly inhibits BKPyV early and late gene expression. DNA viruses and nuclear replicating viruses such as BKPyV rely on host cell processes which in turn rely on MAT2A activity. The results described herein suggest that MAT2A is a promising therapeutic target for the treatment and prevention of viral infections.

### Example 2

Various MAT2A inhibitors (AG270, AZ28, AZ31, AZ32 and AZ33) were tested to identify their potency against BKPyV, and to identify their effects on cell viability. Each of the MAT2A inhibitors tested demonstrated a significant dose dependent inhibition of BKPyV replication at concentrations that had little effect on cell viability and metabolism when added to cells 1 hr post infection (FIGS. 5 and 6A). Early viral protein (LTAg) and late viral protein (VP1) levels were found to be reduced in the presence of each of the MAT2A inhibitors in a dose dependent manner (FIG. 6B). These data demonstrate that various MAT2A inhibitors can be used to significantly inhibit BKPyV early and late gene expression and BKPyV replication.

### Example 3

In addition to MAT2A, TSG101 was identified as a further host factor important for BKPyV infection. Prazoles have been shown to inhibit TSG101 function by targeting the ubiquitin binding domain of TSG101. An exemplary prazole, Ilaprazole, was used to determine whether inhibition of TSG101 affected viral replication and protein expression. Ilaprazole, either alone or in combination with a MAT2A inhibitor, was found to have little effect on cell viability (FIG. 7). Importantly, Ilaprazole was shown to significantly inhibit BKPyV viral replication and protein expression, either alone or in combination with a MAT2A inhibitor (FIG. 8).

MAT2A inhibitors AG270 (1 μM) and AZ33 (125 nM) were shown to have increased potency against BKPyV replication when used in conjunction with the TSG101 inhibitor Ilaprazole (5 μM), with little effect on cell viability and metabolism. At 125 nM, the lowest concentrations used in this study, AZ33 when employed in conjunction with Ilaprazole, showed an almost 100-fold reduction in viral replication titres at 48 hpi, compared to ˜10-fold reduction to viral titres when using AZ33 alone (FIG. 8A). These effects are reflected in both early and late BKPyV protein expression (FIG. 8B).

### Example 4

To confirm that the anti-viral properties of MAT2A are not limited to polyomavirus, the DNA virus Herpes Simplex Virus-1 (HSV-1) was also used. The MAT2A inhibitor PF-9366 (20 μM) demonstrated greater than 100-fold reduction in HSV-1 viral titres (FIG. 9). Interestingly, PF-9366 had little impact on viral protein expression (ICP0 and VP16) indicating that PF-9366 inhibits HSV-1 at a late stage, e.g. virion assembly.

### Example 5

To determine whether RNA viruses are susceptible to MAT2A inhibition, two alphaviruses (Sindbis virus (SINV) and Semliki Forest Virus 4 (SFV4)) and a flavivirus (Zika, American & African strains) were exposed to the MAT2A inhibitor PF-9366. SINV and SFV4 demonstrated a reduction in viral replication when treated with MAT2A inhibitor PF-9366. In initial tests SINV and SFV4, when treated with PF-9366 at 2 μM, both demonstrated about a 7-10-fold reduction in viral titre (FIG. 10A). At similar low doses (2.5 μM) PF-9366 demonstrated about a 2-3-fold reduction in viral titre for BKPyV (FIG. 3B).

In addition, American and African Zika strains demonstrated a reduction in viral protein expression when treated with PF-9366 (20 μM) (FIG. 10B). It is expected that viral protein reduction will directly translate to virus titre reduction.

These data indicate that MAT2A inhibitors can be used to reduce replication of RNA viruses, in addition to DNA viruses.

### Materials and Methods

### Cell Lines, Virus and Antibodies

hTERT Immortalised Renal Proximal Tubule Epithelial Cells (RPTE/TERT1) (ATCC) were cultured in Renal Epithelial Basal Medium (REBM) supplemented with BulletKit (Lonza) or in DMEM/Hams F-12 supplemented with HEPES (10 mM), hEGF (10 ng/ml), Triiodo-L-thyronine (5 pM), L-ascorbic acid (3.5 μg/ml), holo-transferrin (5 μg/ml), prostaglandin E1 (25 ng/ml), hydrocortisone (25 ng/ml), sodium selenite (8.65 ng/ml), insulin (5 μg/ml), G418 (100 μg/ml), 10 μg/ml gentamicin and 0.25 μg/ml amphotericin B and foetal calf serum (FCS) (0.5%). HaCaT, VERO, VERO E6, BHK-21 and BSR cells were cultured in DMEM supplemented with L-Glutamine (2 mM), penicillin (100 U/ml), streptomycin (100 μg/ml) and FCS (10%).

To generate RPTE/TERT1-Cas9 cells RPTE/TERT1 cells were lentiviral transduced with lentiCas9-Blast lentivirus (#52962, Addgene) and selected with blasticidin, generating a cell line constitutively expressing Cas9 (RPTE/TERT1-Cas9). RPTE/TERT1-Cas9 were cultured in REBM supplemented with BulletKit (Lonza) and further supplemented with 10% FCS.

To generate RPTE/TERT1 MAT2A KO cells RPTE/TERT1-Cas9 cells were transduced with Lentivirus encoding MAT2A sgRNA inserted into lentiGuide-Puro plasmid (#52963, Addgene) facilitating MAT2A gene knockout (SH-2 FWD 5′-caccgCCTGATGCCAAAGTAGCTTG (SEQ ID NO: 1), Sh-2 REV 5′-aaacCAAGCTACTTTGGCATCAGGC (SEQ ID NO: 2). Following puromycin selection cells were single cell cloned, clone D3 used in this study was validated as knockout by sequencing. RPTE/TERT1 MAT2A KO cells were cultured in REBM supplemented with BulletKit (Lonza) and further supplemented with 10% FCS.

BK-Dunlop inserted into a pGEM vector (kindly provided by M. Imperiale) was digested using BamHI, then purified and re-ligated. The re-ligated genome was transfected into a T75 flask of RPTE cells in REBM supplemented with BulletKit (Lonza) and further supplemented with 5% FCS. After three weeks, the cells were split into three T150 flasks and left for a further three weeks before harvesting. The cells were freeze thawed three times to release the virus and assayed using a fluorescent focus unit (FFU) assay.

The HSV-1 strain used in this study, SC16 WT HSV-1, is a clinical strain isolated from human oral lesions (Hill et al., J Gen Virol. 1975; 28:341-353). Virus stocks were grown and titrated in VERO cells.

The primary antibodies used were mouse anti-SV40 VP1 (PAb597), rabbit anti-SV40 VP1 (Abcam, ab53977), anti-SV40 T-antigen (Abcam, ab16879), anti-MAT2A/1A (Santa Cruz, sc-166452), anti-VP16 (Abcam, ab110226), anti-ICP0 (Santa Cruz, sc-53070), anti-Zika E Protein, anti-GAPDH (GeneTex, GTX28245), and anti-tubulin [YL1/2] (Abcam, ab6160). Secondary antibodies used for fluorescence were donkey anti-mouse IgG1 and donkey anti-mouse IgG2a conjugated to Alexa Fluor 488 or 568 (Invitrogen). Secondary antibodies used for western blot were goat anti-rat, goat anti-mouse, goat anti-mouse IgG1, or goat anti-mouse IgG2a conjugated to either IRDye 800CW or IRDye 680LT (LI-COR Biosciences).

### Cell Infections and Harvesting

For BKPyV infections, RPTE/TERT1, RPTE/TERT1-Cas9 or RPTE/TERT1 MAT2A KO cells were infected with BKPyV at either a multiplicity of infection (MOI) of 5 for growth curves or an MOI of 3 or 1 for all other experiments, diluted in appropriate medium. At 1 hour post infection (hpi) medium was removed, cells were washed twice with phosphate-buffered saline (PBS), and fresh medium was added with or without inhibitors. At 24, 48, 72 or 96 hpi cells were either harvested for western blot, RT qPCR or virus titre by FFU assay, or fixed for immunofluorescence analysis.

For HSV-1 infections HaCaT cells were infected with HSV-1 at a multiplicity of infection (MOI) of 10 diluted in appropriate medium. At 1 hour post infection (hpi) medium was removed, cells were washed twice with phosphate-buffered saline (PBS), and fresh medium was added with or without inhibitors. At 16 hpi cells were either harvested for western blot or virus titre by plaque assay.

For Alphavirus infections BHK-21 cells were infected with either SINV or SFV-4 at a multiplicity of infection (MOI) greater than 10, diluted in appropriate medium. At 1 hour post infection (hpi) medium was removed, cells were washed twice with phosphate-buffered saline (PBS), and fresh medium was added. At 24 hpi supernatants were harvested for virus titre by plaque assay.

For Flavivirus infections VERO E6 cells were infected with either African (Afn) or American (Amer) Zika at a multiplicity of infection (MOI) greater than 10, diluted in appropriate medium. At 1 hour post infection (hpi) medium was removed, cells were washed twice with phosphate-buffered saline (PBS), and fresh medium was added. At 48 hpi cells were either harvested for western blot.

For western blotting and RT qPCR cells were harvested by centrifugation at 5,000×g after two PBS washes. For FFU assay cells were harvested by scraping up into 1 ml of appropriate media, virus was released from cells by repeated freeze thawing.

### Immunofluorescence Quantification

For fluorescence intensity analysis, 20 fields of view were captured for each experiment. All quantification analyses were carried out using ImageJ and an optimised robust automated detection pipeline Schindelin et al. (Nat Methods. 2012 Jun. 28; 9 (7): 676-82). For each individual experiment, threshold, brightness and contrast were adjusted and standardised to produce comparable levels of accuracy to manual counting. LTAg-staining patterns across each field of view were measured by quantifying the fluorescence intensity using the Mean Grey Value (MGV) function of ImageJ and averaging the results for each experimental condition. Individual cell fluorescence intensity was determined by drawing polygon selections around each LTAg-positive nuclei in ImageJ and measuring the nuclear area, MGV and integrated density, then doing the same for background areas close to each nuclei measured.

Corrected cell fluorescence was measured by the following formula:

$$Integrated Density of nuclei - ( Area of nuclei \times Mean fluorescence of background readings )$$

### Western Blotting and Quantification

Cell lysate preparation and western blots were performed as described in Ren et al. (J Gen Virol. 2012 February; 93 (Pt 2): 319-329). Primary antibodies used were ab53977 (VP1), PAb416 (LTAg), sc-166452 (MAT2A/1A), ab110226 (VP16), sc-53070 (ICP0), anti-Zika E Protein, and either anti-GAPDH or anti-tubulin as a loading control, followed by IRDye 680LT-, or IRDye 800CW-conjugated secondary antibodies. Imaging was performed using the LI-COR Odyssey Infrared Imaging system. Quantification was performed using LI-COR Image Studio Software.

### FFU Assays

FFU assays were used to determine the concentration of infectious BKPyV in experimental samples. RPTE/TERT1 cells were infected with sample dilutions, fixed at 48 hpi in 3% formaldehyde, and immunostained for VP1 expression as described in Evans et al. (Open Biol. 2015 August; 5 (8): 150041).

qPCR

Cells were lysed in 10 μL lysis buffer (Taq PCR buffer without MgCl₂, 300 nM, MgCl₂, 20 mg/mL proteinase K in nuclease-free water). Cells were heated at 60° C. for 1 hr, then at 95° C. for 15 mins, after which 90 μL nuclease-free water was added to dilute the DNA.

Amplification and quantification of BKPyV DNA were performed using the SYBR Green technology protocol. Each experimental condition was measured in technical triplicate. Using a PCR 96-well plate, the amplification reactions were carried out in a total volume of 25 μL, (5 μL of template DNA, 12.5 μL of 2× qPCR MasterMix for SYBR Green I No ROX, 0.25 μL each of (10 μM) forward and reverse primer, and 7 μL of nuclease-free water). Primers for the BKPyV genome were designed and obtained through TIB MolBiol and targeted a region in the agnoprotein gene (FWD: TGTCACGWMARGCTTCWGTGAAAG TT (SEQ ID NO: 3); REV: AGAGTCTITTACAGCAGGTAAAGCAG (SEQ ID NO: 4)).

Delta-delta CT (2-ΔΔCt) method was performed to evaluate the relative viral DNA copy number using GAPDH as a reference housekeeping gene.

### Plaque Assays

Plaque assays were used to determine the concentration of infectious HSV-1 and Alphaviruses in experimental samples.

For HSV-1, confluent VERO cell monolayers were infected with 10-fold dilutions of samples. At 1 hpi cells were overlayed with DMEM supplemented with L-Glutamine (2 mM), penicillin (100 U/ml), streptomycin (100 μg/ml), 0.3% high viscosity carboxymethyl cellulose, and 0.3% low viscosity carboxymethyl cellulose. Cells were fixed at 48 hpi and stained with toluidine blue, allowing plaques to be counted.

For SINV and SFV4, confluent BSR cell monolayers were infected with 10-fold dilutions of samples. At 1 hpi cells were overlayed with low melting-point agarose in 2×MEM (+5% FCS). Cells were fixed at 72 hpi and stained with toluidine blue, allowing plaques to be counted.

### Inhibitors

Example 1: PF-9366 (Sigma-Aldrich) and AG-270 (kindly supplied by Astra Zeneca) were diluted in 100% DMSO. Cells were drug treated at a range of concentrations in REBM supplemented with BulletKit (Lonza) for 3 hours prior to infection. Cells were infected with BK-Dunlop in media and appropriate drug dilution. At 1 hpi infectious media was removed, cells gently washed with PBS and media containing appropriate drug dilutions was added. At 48 hpi cells were either fixed in 3% formaldehyde for microscopy, or harvested by scraping up into fresh drug-free media.

Examples 2-5: PF-9366 (Sigma-Aldrich) and AG-270, AZ28, AZ31, AZ32 and AZ33 (kindly supplied by Astra Zeneca) were diluted in 100% DMSO. TSG101 inhibitor Ilaprazole (Sigma-Aldrich) was diluted in 100% DMSO. Cells were treated at a range of inhibitor concentrations and combinations, in appropriate media for the cell type used, for either 3 hours prior to infection (HSV-1, Alphavirus and Flavivirus infections) or 1 hour post infection (BKPyV infection).

### Cell Viability Assay

Cell viability was assessed using the Cell-Titer-Blue assay (Promega), following manufacturers guidelines.

### Error Bars and Statistics

Where possible (a minimum of 3 experiments conducted), one-sample t tests were conducted to give P values (standard deviations are shown with error bars).

1. A MAT2A inhibitor and/or a TSG101 inhibitor for use in a method of treating or preventing a viral infection in a subject.

2. A method for treating or preventing a viral infection in a subject, the method comprising administering to the subject a MAT2A inhibitor and/or a TSG101 inhibitor.

3. The MAT2A inhibitor and/or the TSG101 inhibitor for use according to claim 1, wherein the viral infection is caused by a DNA virus.

4. The MAT2A inhibitor and/or the TSG101 inhibitor for use according to claim 3, wherein the DNA virus is a double stranded DNA virus.

5. The MAT2A inhibitor and/or the TSG101 inhibitor for use according to claim 4, wherein the DNA virus is selected from a polyomavirus, a papillomavirus, an adenovirus, a poxvirus, or a herpesvirus.

6. The MAT2A inhibitor and/or the TSG101 inhibitor for use according to claim 5, wherein the DNA virus is a polyomavirus.

7. The MAT2A inhibitor and/or the TSG101 inhibitor for use according to claim 6, wherein the DNA virus is BK polyomavirus or JC polyomavirus.

8. The MAT2A inhibitor and/or the TSG101 inhibitor for use according to claim 7, wherein the method comprises treating or preventing polyomavirus associated nephropathy.

9. The MAT2A inhibitor and/or the TSG101 inhibitor for use according to claim 7, wherein the method comprises treating or preventing progressive multifocal leukoencephalopathy.

10. The MAT2A inhibitor and/or the TSG101 inhibitor for use according to claim 7, wherein the method comprises treating or preventing late-onset haemorrhagic cystitis.

11. The MAT2A inhibitor and/or the TSG101 inhibitor for use according to claim 5, wherein the DNA virus is a papillomavirus.

12. The MAT2A inhibitor and/or the TSG101 inhibitor for use according to claim 1, wherein the viral infection is caused by a nuclear replicating virus.

13. The MAT2A inhibitor and/or the TSG101 inhibitor for use according to claim 12, wherein the nuclear replicating virus is a retrovirus, optionally wherein the retrovirus is HIV.

14. The MAT2A inhibitor and/or the TSG101 inhibitor for use according to claim 12, wherein the nuclear replicating virus is an influenza virus.

15. The MAT2A inhibitor and/or the TSG101 inhibitor for use according to claim 1, wherein the viral infection is caused by an RNA virus, optionally wherein the RNA virus is selected from an alphavirus, an astrovirus, a calicivirus, a flavivirus, and a picornavirus.

16. The MAT2A inhibitor and/or the TSG101 inhibitor for use according to claim 1, wherein the viral infection is caused by a non-enveloped virus.

17. The MAT2A inhibitor and/or the TSG101 inhibitor for use according to claim 1, wherein the subject is immunosuppressed.

18. The MAT2A inhibitor and/or the TSG101 inhibitor for use according to claim 1, wherein the subject is a transplant patient.

19. The MAT2A inhibitor and/or the TSG101 inhibitor for use according to claim 18, wherein the subject is a kidney transplant patient.

20. The MAT2A inhibitor and/or the TSG101 inhibitor for use according to claim 18, wherein the subject is a haematopoietic stem cell transplant patient.

21. The MAT2A inhibitor and/or the TSG101 inhibitor for use according to claim 1, wherein the MAT2A inhibitor is selected from a small molecule, a peptide, a cyclic peptide, and a peptidomimetic.

22. The MAT2A inhibitor and/or the TSG101 inhibitor for use according to claim 1, wherein the MAT2A inhibitor is:

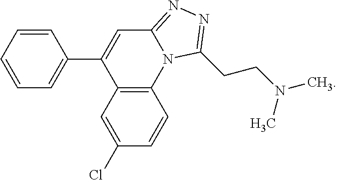 [SMILES: CN(C)CCc1nnc2cc(-c3ccccc3)c3cc(Cl)ccc3n12]

23. The MAT2A inhibitor and/or the TSG101 inhibitor for use according to claim 1, wherein the MAT2A inhibitor is:

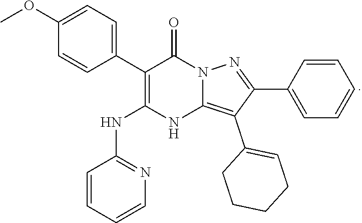 [SMILES: COc1ccc(-c2c(Nc3ccccn3)[nH]c3c(C4=CCCCC4)c(-c4ccccc4)nn3c2=O)cc1]

24. The MAT2A inhibitor and/or the TSG101 inhibitor for use according to claim 1, wherein the MAT2A inhibitor is:

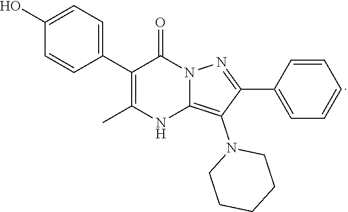 [SMILES: Cc1[nH]c2c(N3CCCCC3)c(-c3ccccc3)nn2c(=O)c1-c1ccc(O)cc1]

25. The MAT2A inhibitor and/or the TSG101 inhibitor for use according to claim 1, wherein the MAT2A inhibitor is:

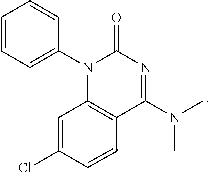 [SMILES: CN(C)c1nc(=O)n(-c2ccccc2)c2cc(Cl)ccc12]

26. The MAT2A inhibitor and/or the TSG101 inhibitor for use according to claim 1, wherein the MAT2A inhibitor is: 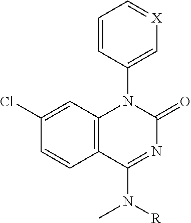
wherein R is Me or Et and X is C or N.

27. The MAT2A inhibitor and/or the TSG101 inhibitor for use according to claim 1, wherein the MAT2A inhibitor is IDE397.

28. The MAT2A inhibitor and/or the TSG101 inhibitor according to claim 1, wherein the MAT2A inhibitor is an oligonucleotide, optionally wherein the MAT2A inhibitor is an antisense oligonucleotide, a shRNA, a siRNA, a microRNA, or an aptamer.

29. The MAT2A inhibitor and/or the TSG101 inhibitor according to claim 1, wherein the MAT2A inhibitor is an antibody.

30. The MAT2A inhibitor and/or the TSG101 inhibitor for use according to claim 1, wherein the TSG101 inhibitor is selected from a small molecule, a peptide, a cyclic peptide, and a peptidomimetic.

31. The MAT2A inhibitor and/or the TSG101 inhibitor for use according to claim 1, wherein the TSG101 inhibitor is a prazole.

32. The MAT2A inhibitor and/or the TSG101 inhibitor according to claim 1, wherein the TSG101 inhibitor is selected from Ilaprazole, omeprazole, esomeprazole, dexlansoprazole, lansoprazole, pantoprazole, rabeprazole, and tenatoprazole.

33. The MAT2A inhibitor and/or the TSG101 inhibitor for use according to claim 1, wherein the TSG101 inhibitor is selected from an oligonucleotide, optionally wherein the TSG101 inhibitor is an antisense oligonucleotide, a shRNA, a siRNA, a microRNA, or an aptamer.

34. The MAT2A inhibitor and/or a TSG101 inhibitor according to claim 1, wherein the TSG101 inhibitor is an antibody.

35. Use of a MAT2A inhibitor and/or a TSG101 inhibitor in the treatment or prevention of a viral infection in a subject.

36. The use according to claim 35, wherein the viral infection is caused by a DNA virus.

37. The use according to claim 35, wherein the viral infection is caused by a nuclear replicating virus.

38. The use according to claim 35, wherein the viral infection is caused by an RNA virus, optionally wherein the RNA virus is selected from an alphavirus, an astrovirus, a calicivirus, a flavivirus, and a picornavirus.

39. The use according to claim 35, wherein the viral infection is caused by a non-enveloped virus, optionally wherein the non-enveloped virus is selected from a polyomavirus, a papillomavirus, an adenovirus, a parvovirus, an astrovirus, a calicivirus, a picornavirus, or a reovirus.


SEQUENCE LISTING (4 sequences)

SEQ ID NO: 1 - DNA, 25 bp, synthetic construct
  source 1..25: mol_type = other DNA
caccgcctgatgccaaagtagcttg

SEQ ID NO: 2 - DNA, 25 bp, synthetic construct
  source 1..25: mol_type = other DNA
aaaccaagctactttggcatcaggc

SEQ ID NO: 3 - DNA, 26 bp, synthetic construct
  source 1..26: mol_type = other DNA
tgtcacgwmargcttcwgtgaaagtt

SEQ ID NO: 4 - DNA, 26 bp, synthetic construct
  source 1..26: mol_type = other DNA
agagtcttttacagcaggtaaagcag